(19) United States
(12) Patent Application Publication
(10) Pub. No.: US 2023/0233198 A1
(43) Pub. Date: Jul. 27, 2023

(54) PARTITIONING ELEMENT FOR PARTITIONING A LUMEN, AND A METHOD FOR SEALABLY ISOLATING A FIRST PORTION OF A LUMEN FROM A SECOND PORTION THEREOF AND FOR INSUFFLATING THE FIRST PORTION OF THE LUMEN

(71) Applicant: PALLIARE LIMITED, Galway (IE)

(72) Inventor: John O'DEA, Galway (IE)

(73) Assignee: PALLIARE LIMITED, Galway (IE)

(21) Appl. No.: 18/156,777

(22) Filed: Jan. 19, 2023

(30) Foreign Application Priority Data
Jan. 21, 2022 (IE) S2022/0014
Jul. 12, 2022 (IE) S2022/0115

(51) Int. Cl. A61B 17/02

(52) U.S. Cl. CPC A61B 17/0218; A61B 2017/00477

(57) ABSTRACT

A pair of partitioning elements located in a duodenum, sealably isolates a first portion of the duodenum, in which a minimally invasive procedure is to be carried out from a proximal second portion and a distal third portion of the duodenum to allow insufflating of the first portion only. The first partitioning element located between the first and second portions of the duodenum comprises a tubular carrier element and an isolating membrane secured to the carrier element. The carrier element is radially expandable from a compressed first state to a second state with the isolating membrane sealably isolating the first portion from the second portion. A communicating opening through the isolating membrane accommodates an endoscope therethrough into the first portion. The communicating opening is omitted from the second partitioning element.

### FIELD OF THE INVENTION

The present invention relates to a partitioning element for substantially sealably isolating a first portion of a lumen from a second portion thereof, for example, for substantially sealably isolating a first portion of a lumen in the body of a human or animal subject from a second portion thereof. The invention also relates to a method for substantially sealably isolating a first portion of a lumen from a second portion of the lumen, for example, a lumen in the body of a human or animal subject. Additionally, the invention relates to a method for insufflating a first portion of a lumen independently of a second portion thereof or independently of the remainder of the lumen, such as a lumen in the body of a human or animal subject.

### BACKGROUND TO THE INVENTION

Investigative and surgical procedures are regularly required to be carried out in a short portion of an elongated lumen in the body of a human or animal subject, for example, the duodenum, the colon, the oesophagus and other such lumens. In the carrying out of such procedures by minimally invasive surgical procedures, it is necessary to insufflate the lumen in the location of the site at which the procedure is to be carried out. Unfortunately, in an elongated lumen insufflating gas flows from the location being insufflated to the remainder of the lumen. Thus, in order to insufflate a location in an elongated lumen, the entire lumen becomes insufflated. It has been found that insufflating of an entire lumen may cause post-operative pain in a subject, due to the stretching of the entire lumen by insufflating thereof. Attempts have been made to address this problem by providing partitioning elements for placing in the lumen at either end of the portion of the lumen which is to be insufflated, to thereby isolate the portion to be insufflated from the remainder of the lumen. However, such partitioning elements known heretofore require support systems which must pass through the portion of the lumen being insufflated in order to maintain the partitioning elements active to isolate the portion of the lumen being insufflated from the remainder of the lumen. Such support systems may, for example, include inflating conduits in order to maintain the partitioning elements inflated. The provision of such support systems passing through the portion of the lumen being insufflated results in considerable inconvenience and can hinder the carrying out of the procedure in the insufflated portion of the lumen.

There is therefore a need for a partitioning element for isolating, and preferably, substantially sealably isolating a portion of a lumen from another portion of the lumen or from the remainder of the lumen, which addresses this problem, and in particular, there is a need for such a partitioning element which is easily positioned in the lumen and easily removable from the lumen for temporarily substantially sealably isolating a portion of the lumen in which a minimally invasive procedure is to be carried out from an adjacent portion of the lumen or from the remainder of the lumen.

The present invention is directed towards providing such a partitioning element, and the invention is also directed towards providing a method for substantially sealably isolating a first portion of a lumen from a second portion thereof. Additionally, the invention provides a method for insufflating a first portion of a lumen independently of a second portion thereof or independently of the remainder of the lumen, and the invention is also directed towards a lumen comprising one or a pair of partitioning elements with a portion of the lumen sealably isolated from the remainder of the lumen.

Throughout this specification and the claims thereof, the terms “proximal” and “distal” are used to identify the part of a lumen, a component or a partitioning element which, in use, is proximal or distal relative to a surgeon or clinician carrying out the procedure. The term “proximal” being used to indicate that part of the lumen, component or partitioning element which is closest to the surgeon or clinician in use, and the term “distal” being used to indicate that part of the lumen, component or partitioning element which is furthest away from the surgeon or clinician. Additionally, the term “lumen” as used in this specification and the claims thereof, is intended to mean any lumen, vessel or organ in which a portion of the lumen, vessel or organ is to be isolated from another portion thereof.

### SUMMARY OF THE INVENTION

According to the invention there is provided a partitioning element for isolating a first portion of an elongated lumen from a second portion thereof, the partitioning element comprising:
an expandable carrier element for locating in the lumen between the first and second portions thereof, the carrier element extending from a proximal end to a distal end and having a bore extending longitudinally therethrough and being radially expandable from a first state defining a first transverse cross-sectional area to a second state defining a second transverse cross-sectional area greater than the first transverse cross-sectional area,
an isolating element carried on and secured to the carrier element and being urgeable from a non-isolating state to an isolating state with one of the isolating element and the carrier element engaging an inner surface of the lumen and the isolating element extending transversely across the lumen to substantially sealably isolate the first portion of the lumen from the second portion thereof as the carrier element expands from the first state thereof to the second state, and
a connector adapted to be releasably or non-releasably connectable with an operating element for withdrawing the partitioning element from the lumen, the connector being formed by a part of the carrier element or by a part of the isolating element or being connected either to the isolating element or to at least two lanyards secured to the periphery of the carrier element at circumferentially spaced apart intervals adjacent the proximal end thereof.

In one embodiment of the invention the carrier element is of tubular construction defining the longitudinally extending bore.

In another embodiment of the invention the isolating element is secured to the carrier element adjacent the proximal end of the carrier element.

In an alternative embodiment of the invention the isolating element is secured to the carrier element adjacent the distal end of the carrier element.

In a further alternative embodiment of the invention the isolating element is secured to the carrier element intermediate the proximal and distal ends of the carrier element.

In another embodiment of the invention the isolating element is secured to the carrier element adjacent a periphery of the carrier element, and preferably, the isolating element is secured to the carrier element around the periphery thereof. Preferably, the one of the isolating element and the carrier element is adapted to engage an inner surface of the lumen with substantially sealable engagement.

Preferably, one or both of the isolating element and the at least two lanyards are secured to the carrier element, so that when the distal end of the carrier element is engaged in the lumen, by urging the connector proximally the transverse cross-sectional area defined by the carrier element commences to reduce from the proximal end thereof towards the distal end thereof.

In one embodiment of the invention the one of the isolating element and the at least two lanyards are secured to the carrier element, so that when the distal end of the carrier element is stationary, by urging of the connector proximally by the operating element reduces the transverse cross-sectional area defined by the carrier element in the second state thereof adjacent the proximal end, and preferably, progressively reduces the transverse cross-sectional area defined by the carrier element from the distal end thereof to the proximal end thereof.

In one embodiment of the invention the connector comprises a part of the isolating element, and preferably, the connector comprises a part of the isolating element adjacent a central portion thereof.

In another embodiment of the invention the part of the isolating element forming the connector is gathered to form the connector, and preferably, the part of the isolating element forming the connector is gathered to form the connector as a connecting tab.

In another embodiment of the invention the connector is connected to the at least two lanyards, and preferably, the connector comprises one of a connecting loop, a connecting ring, and a connecting tab.

In another embodiment of the invention the connector is formed by free ends of the lanyards.

Preferably, the lanyards are connected to the carrier element at equi-spaced apart intervals around the carrier element.

In another embodiment of the invention the carrier element comprises a memory metal, and preferably, the carrier element is configured to be unstable in the first state thereof and to be inherently urgeable from the first state thereof to the second state.

In another embodiment of the invention the carrier element is adapted to be restrained in the first state, and to expand to the second state when unrestrained.

In one embodiment of the invention the carrier element is configured to remain in the expanded second state without an external support.

In one embodiment of the invention the carrier element comprises a tubular element of stent type construction.

In another embodiment of the invention the carrier element comprises a plurality of struts defining interstices therebetween, and preferably, each strut extends in a generally longitudinal circumferential direction.

In another embodiment of the invention the carrier element is woven, and preferably, is woven from strands of a memory metal.

In another embodiment of the invention the carrier element is formed from a tubular element by slotting and expanding the tubular element.

In one embodiment of the invention the carrier element is expanded to the second state thereof and is temperature set in the second state thereof.

In one embodiment of the invention the carrier element is of circular transverse cross-section, and preferably, is of cylindrical shape.

In one embodiment of the invention the isolating element is adapted to engage the inner surface of the lumen in the isolating state thereof with substantially sealing engagement.

In another embodiment of the invention the isolating element comprises a membrane, and preferably, a membrane of material substantially impermeable to an insufflating gas, for example, substantially impermeable to carbon dioxide or air.

In another embodiment of the invention the membrane comprises a sheet of material, and advantageously, a sheet of flexible material.

In one embodiment of the invention the isolating element comprises a resilient material, and in an alternative embodiment of the invention the isolating element comprises a non-resilient material.

In another embodiment of the invention the isolating element is secured to an outer surface defined by the carrier element.

In one embodiment of the invention the isolating element is secured to the carrier element by an adhesive, and in an alternative embodiment of the invention the isolating element is secured to the carrier element by stitching, suturing or fastening.

In a further embodiment of the invention the isolating element is foldable in the non-isolating state, and preferably, the isolating element in the non-isolating state is located within the carrier element when the carrier element is in the first state thereof.

In another embodiment of the invention a communicating opening is formed through the isolating element for accommodating one of an instrument, an insufflating gas delivery conduit and an endoscope therethrough, and in another embodiment of the invention the isolating element adjacent the communicating opening is adapted to engage a portion of an instrument extending therethrough or the endoscope extending therethrough, and preferably, is adapted to slidably engage a portion of an instrument or an endoscope, and advantageously, the isolating element adjacent the communicating opening is adapted to engage a portion of the instrument or an endoscope with substantially sealing engagement.

In another embodiment of the invention a delivery element is provided for delivering the partitioning element to the location at which the first portion of the lumen is to be isolated from the second portion thereof, and preferably, the delivery element is configured to restrain the carrier element in the first state thereof.

In another embodiment of the invention the operating element is cooperable with the delivery element for discharging the partitioning element in the lumen adjacent the location between the first and second portions thereof.

Preferably, the operating element is configured to retain the partitioning element in position adjacent the location between the first and second portions of the lumen while the delivery element is being withdrawn proximally for discharging the partitioning element from the delivery element.

In another embodiment of the invention the delivery element comprises a delivery catheter, and preferably, the partitioning element is located in the delivery catheter, and preferably, the partitioning element is located in the delivery catheter adjacent a distal end thereof, and preferably, the operating element extends through the delivery catheter from a proximal end of the catheter to the partitioning element, and preferably, the operating element is releasably coupled to the connector of the partitioning element with the partitioning element adjacent the distal end of the delivery catheter.

In another embodiment of the invention the operating element is cooperable with the delivery element for withdrawing the partitioning element from the lumen, and preferably, the operating element is cooperable with the delivery element for drawing the partitioning element into the delivery element from the lumen.

In another embodiment of the invention the connector is configured to be gripped by a gripper of the operating element, and preferably, the connector is configured to be gripped between a pair of jaws of a gripper of the operating element.

In one embodiment of the invention the connector is configured to be urgeable by the operating element for urging the partitioning element distally in the lumen for placing the partitioning element at the location in the lumen between the first and second portions thereof. Alternatively, the connector is configured to be held by the operating element while the delivery element is being urged proximally for discharging the partitioning element from the delivery element into the lumen.

In one embodiment of the invention the operating element is provided, and the operating element is configured for urging the partitioning element through the lumen to the location at which it is to be placed, and preferably, the operating element is configured for withdrawing the partitioning element from the location in the lumen at which it had been placed.

In another embodiment of the invention the operating element is configured for urging the partitioning element proximally relative to the delivery element while the delivery element is held stationary in the lumen or is being urged distally in the lumen for drawing the partitioning element into the delivery element, and preferably, for progressively drawing the partitioning element into the delivery element. Preferably, the operating element is configured to urge the connector of the partitioning element proximally as the delivery element is being urged distally for drawing the partitioning element into the delivery element.

In another embodiment of the invention a pair of the partitioning elements are provided, a first one of the partitioning elements being configured to isolate the first portion of the lumen from the second portion thereof, and a second one of the partitioning elements being configured to isolate the first portion of the lumen from a third portion thereof remote from the second portion of the lumen.

In another embodiment of the invention the first one of the partitioning elements is provided with the communicating opening extending through the isolating element, and the second one of the partitioning elements is provided without a communicating opening extending through the isolating element, and preferably, the partitioning elements are located spaced apart in the lumen defining the first portion thereof therebetween.

Further the invention provides a partitioning element for substantially sealably isolating a first portion of an elongated lumen from a second adjacent portion of the lumen, the partitioning element comprising a carrier element, and an isolating element carried on the carrier element, one of the carrier element and the isolating element having an outer periphery defining an outer periphery of the partitioning element engageable with an inner surface of a wall of the lumen, one of the carrier element and the isolating element being expandable from a non-expanded first state with the outer periphery of the partitioning element defining an area of a first size to an expanded second state with the outer periphery of the partitioning element defining an area of a second size greater than the area of the first size for sealably engaging the inner surface of the wall of the lumen to substantially sealably isolate the first portion of the lumen from the adjacent second portion thereof.

In one aspect of the invention the expandable one of the carrier element and the isolating element comprises a non-inflatable element.

Preferably, the expandable one of the carrier element and the isolating element is configured to remain in the expanded second state without external support.

In one embodiment of the invention a connector is located on one of the carrier element and the isolating element, and preferably, is releasably engageable by an operating element for positioning the partitioning element in the lumen prior to expansion of the one of the carrier element and the isolating element from the first state to the second state thereof.

In another embodiment of the invention the connector is adapted to be disengaged from the operating element once the partitioning element has been positioned and the expandable one of the carrier element and the isolating element has been expanded from the first state to the second state.

In one embodiment of the invention a delivery means is provided for delivering the partitioning element to a location at which the partitioning element is to be deployed.

In one embodiment of the invention the delivery means is configured to maintain the expandable one of the carrier element and the isolating element in the first unexpanded state.

In one embodiment of the invention a communicating opening extends through one of the carrier element and the isolating element for accommodating an instrument or an endoscope therethrough into the first portion of the lumen, and in another embodiment of the invention the communicating opening is adapted for accommodating the instrument or the endoscope from the second portion of the lumen into the first portion thereof.

In another embodiment of the invention the communicating opening is adapted to sealably or at least partially sealably engage the instrument or the endoscope.

In another embodiment of the invention a pair of the partitioning elements are provided, a first one of the partitioning elements being provided for isolating the first portion of the lumen from the second portion thereof, and a second one of the partitioning elements being provided for isolating the first portion of the lumen from a third portion thereof remote from the second portion thereof. Preferably, the first portion of the lumen is located between the second and third portions thereof. Advantageously, the second portion of the lumen is located proximally of the first portion thereof, and preferably, the first one of the pair of the partitioning elements is provided with the communicating opening, and the second one of the partitioning elements does not include the communicating opening.

In one embodiment of the invention the carrier element comprises a biologically compatible material.

In another embodiment of the invention the isolating element comprises a biologically compatible material.

In one embodiment of the invention the carrier element is expandable from the unexpanded first state to the expanded second state, and preferably, the isolating element comprises an isolating membrane carried on the carrier element, and the isolating membrane is configured to extend transversely across the lumen to partition the first portion of the lumen from the second portion thereof on expansion of the carrier element from the first state thereof to the second state thereof.

In another embodiment of the invention the isolating membrane is located on the carrier element, so that when the carrier element expands from the first state to the second state thereof, the isolating membrane sealably engages the inner surface of the wall of the lumen.

In one aspect of the invention the isolating membrane comprises a flexible membrane, and in another aspect of the invention the isolating membrane comprises a non-elastic membrane, while in an alternative aspect of the invention the isolating membrane comprises an elastic membrane.

Preferably, the isolating membrane is foldable into a size which does not exceed the area of the first size defined by the outer periphery of the carrier element in the first state thereof.

Preferably, the isolating membrane is impermeable to an insufflating gas with which the first portion of the lumen is to be insufflated.

In one embodiment of the invention the communicating opening extends through the isolating membrane.

In another embodiment of the invention the carrier element comprises a memory metal, and preferably, the carrier element is of stent like construction.

In another embodiment of the invention the delivery means comprises a tubular delivery element having a bore extending longitudinally therethrough for delivering the partitioning element to the location in the lumen in which the partitioning element is to be deployed therein, the tubular delivery element being configured to retain the carrier element in the first unexpanded state therein.

In one embodiment of the invention the delivery element is configured to accommodate the partitioning element in the bore thereof adjacent the distal end of the delivery element.

In another embodiment of the invention the operating element is configured to retain the partitioning element in the position in which the partitioning element is to be deployed in the lumen while the carrier element is being expanded, and preferably, the carrier element is expanded by disengaging the delivery element from the partitioning element. Advantageously, the operating element is configured to retain the partitioning element in position in the lumen, while the delivery element is being withdrawn from the lumen to accommodate expansion of the carrier element from the first state to the second state.

In another embodiment of the invention the isolating membrane has an outer periphery, which preferably, is secured to an outer peripheral rim of the carrier element.

In another embodiment of the invention the connector extends from the carrier element and defines an eye engageable with a hook of the operating element.

In one embodiment of the invention the operating element is configured to draw the carrier element into the delivery element for urging the carrier element from the expanded second state to the unexpanded first state for removal of the partitioning element from the lumen.

In another embodiment of the invention the isolating element comprises an expandable element, and preferably, the isolating element comprises an expandable material located around a circumferential periphery of the carrier element.

In one embodiment of the invention the expandable material is housed in a membrane secured to the carrier element, and preferably, the expandable material of the isolating element comprises an expandable material sold under the trade name Expancel by Nouryon Inc.

In another embodiment of the invention the membrane housing the expandable material is burstable on expansion of the expandable material.

In another embodiment of the invention the expandable material is expandable by an application of heat thereto, and preferably, the carrier element comprises a heat conductive material for conducting heat to the expandable material from a heating element applied thereto.

In another embodiment of the invention the carrier element comprises a non-expandable member, preferably, of circular transverse cross section.

In another embodiment of the invention the carrier element comprises a tubular member having a bore extending therethrough, and advantageously the bore is closed by a disc element.

In one embodiment of the invention the communicating opening extends through the carrier element, and preferably, through the disc element closing the bore of the tubular carrier element.

In another embodiment of the invention the connector extends from the carrier element.

In one embodiment of the invention the carrier element comprises a stainless-steel material.

In one embodiment of the invention the partitioning element is configured for substantially sealably isolating the first portion of the lumen from the second portion thereof to enable insufflating of the first portion of the lumen independently of the second portion thereof.

In another embodiment of the invention a pair of the partitioning elements is provided, a first one of which partitioning elements comprises the communicating opening and is configured for substantially sealably isolating the first portion of the lumen from the second portion thereof, and a second one of the partitioning elements substantially sealably isolates the first portion of the lumen from a third portion thereof remote from the second portion thereof. Preferably, the second and third portions of the lumen are at respective opposite ends of the first portion for enabling insufflating of the first portion of the lumen independently of the second and third portions thereof.

Preferably, the partitioning element having the communicating opening extending therethrough is located in the lumen proximally relative to the partitioning element without the communicating opening, the proximal end of the lumen being the end of the lumen through which an instrument or an endoscope is delivered to the first portion of the lumen through the communicating opening of the proximally located partitioning element.

The invention also provides a method for substantially sealably isolating a first portion of a lumen from a second portion thereof for insufflating the first portion of the lumen independently of the second portion thereof, the method comprising delivering the partitioning element according to the invention to the lumen in the first state thereof and locating the partitioning element in the lumen at a location between the first and second portions of the lumen, expanding the expandable one of the carrier element and the isolating element for engaging the partitioning element with an inner surface of the wall of the lumen.

Additionally, the invention provides a method for substantially sealably isolating a first portion of an elongated lumen from a second portion thereof, the method comprising placing a partitioning element according to the invention with the carrier element in the first state thereof in the lumen adjacent a location between the first and second portions thereof and with the proximal end of the carrier element proximal of the distal end, expanding the carrier element from the first state thereof with the partitioning element extending substantially transversely across the lumen adjacent the location between the first portion of the lumen and the second portion thereof until the one of the isolating element and the carrier element is engaging the inner surface of the lumen with the first portion of the lumen substantially sealably isolated from the second portion thereof.

In one embodiment of the invention the partitioning element is delivered to the lumen by one of the delivery element and the operating element, and preferably, by both the delivery element and the operating element cooperating with each other.

In another embodiment of the invention the connector of the partitioning element is engaged by the operating element during delivery of the partitioning element to the lumen.

In another embodiment of the invention the partitioning element is delivered to the lumen in the delivery element, and preferably, the partitioning element is delivered to the lumen in the delivery element with the operating element engaged with the connector of the partitioning element.

Preferably, the partitioning element is located adjacent the proximal end of the first portion of the lumen by the delivery element.

In one embodiment of the invention with the partitioning element positioned adjacent the proximal end of the first portion of the lumen by the operating element, the delivery element is withdrawn from the partitioning element, and preferably, the expandable one of the carrier element and the isolating element expands on withdrawal of the delivery element therefrom, and preferably, the partitioning element is retained in position at the location between the first and second portions of the lumen by the operating element while the delivery element is withdrawn, and advantageously, on expansion of the expandable one of the carrier element and the isolating element from the first state to the second state thereof, the operating element is disengaged from the connector and is withdrawn from the lumen, and preferably, withdrawn from the lumen with the delivery element.

In an alternative embodiment of the invention once the partitioning element is located at the location between the first and second portions of the lumen, the expandable one of the carrier element and the isolating element is expanded from the unexpanded first state to the expanded second state, and in one embodiment of the invention the expandable one of the carrier element and the isolating element is expanded from the first state to the second state by applying heat to the expandable one of the carrier element and the isolating element.

In one embodiment of the invention the heat is applied to the carrier element for transfer to the isolating element.

In another embodiment of the invention the heat is applied to the one of the carrier element and the isolating element by a heating element, and advantageously, the heating element is delivered through an endoscope.

In one embodiment of the invention the one of the partitioning element which is located adjacent the distal end of the first portion of the lumen is provided without the communicating opening.

In one embodiment of the invention a pair of the partitioning elements are sequentially delivered to the lumen, the partitioning element to be located adjacent the distal end of the first portion of the lumen being delivered to the lumen before the partitioning element to be located adjacent the proximal end of the first portion of the lumen. Preferably, the partitioning element to be located adjacent the proximal end of the first portion of the lumen comprises the communicating opening.

The invention also provides a pair of the partitioning elements according to the invention, one of the partitioning elements comprising the communicating opening, and the other one of the partitioning elements being provided without the communicating opening.

Further, the invention provides a method for isolating a first portion of an elongated lumen from a second portion thereof, the method comprising:
providing a partitioning element comprising:
   an expandable carrier element extending from a proximal end to a distal end and having a bore extending longitudinally therethrough and being radially expandable from a first state defining a first transverse cross-sectional area to a second state defining a second transverse cross-sectional area greater than the first transverse cross-sectional area,
   an isolating element carried on and secured to the carrier element and being urgeable from a non-isolating state to an isolating state as the carrier element expands from the first state thereof to the second state thereof, and
   a connector formed by a part of the carrier element or by a part of the isolating element or being connected either to the isolating element or to at least two lanyards secured to the periphery of the carrier element at circumferentially spaced apart intervals adjacent the proximal end thereof, the method further comprising:
placing the partitioning element in the lumen at a location between the first and second portions thereof with the carrier element in the first state thereof and the proximal end of the carrier element located adjacent the second portion of the lumen, and
radially expanding the carrier element from the first state thereof until the one of the isolating element and the carrier element engages an inner surface of the lumen with the isolating element extending transversely across the lumen substantially sealably isolating the first portion thereof from the second portion thereof.

In one embodiment of the invention the method further comprises delivering the partitioning element by a delivery element to the location in the lumen between the first and second portions thereof with the partitioning element located in the delivery element, and discharging the partitioning element from the delivery element.

In another embodiment of the invention the method further comprises retaining the partitioning element at the location in the lumen between the first portion and the second portion thereof by an operating element connected to the connector of the partitioning element, as the partitioning element is being discharged from the delivery element.

In another embodiment of the invention the partitioning element is discharged from the delivery element by urging the delivery element proximally in the lumen relative to the partitioning element.

In another embodiment of the invention the partitioning element is drawn proximally into the delivery element by the operating element with the operating element connected to the connector for removal of the partitioning element from the lumen.

In one embodiment of the invention the operating element is urged progressively proximally into the delivery element for reducing the transverse cross-sectional area of the carrier element from the proximal end thereof towards the distal end thereof.

Preferably, the connector is urged proximally by the operating element for progressively reducing the transverse cross-sectional area defined by the carrier element from the proximal end thereof to the distal end thereof.

Preferably, a pair of the partitioning elements are placed in the lumen to isolate the first portion of the lumen from the second portion thereof and from a third portion thereof remote from the second portion thereof, a first one of the partitioning elements being located in the lumen between the first and second portions thereof for isolating the first portion of the lumen from the second portion thereof, and a second one of the partitioning elements being located between the first portion thereof and the third portion thereof for isolating the first portion of the lumen from the third portion thereof.

In another embodiment of the invention the method comprises delivering the partitioning element by a delivery element to the location at which the partitioning element is to be deployed in the lumen, and preferably, the carrier element is restrained by the delivery element in the first state thereof, and advantageously, the partitioning element is located in the delivery means, and preferably, the partitioning element is located in the delivery element adjacent a distal end thereof. Preferably, the delivery element is urged through the lumen until the distal end of the delivery element is located adjacent the location at which the partitioning element is to be deployed in the lumen.

In another embodiment of the invention the operating element is cooperable with the delivery element for discharging the partitioning element from the delivery element.

In another embodiment of the invention the partitioning element is retained in position at the location at which the partitioning element is to be deployed in the lumen by the operating element, while the delivery element is being urged proximally in the lumen for discharging the partitioning element from the delivery means into the lumen.

In another embodiment of the invention the operating element is cooperable with the delivery element for drawing the partitioning element from the lumen into the delivery element.

In another embodiment of the invention the connector is gripped by a gripper of the operating element, and preferably, the operating element comprises a gripper element. Preferably, the gripper element comprises a pair of jaws, and the connector is gripped between the jaws of the gripper element.

In another embodiment of the invention the delivery element comprises a delivery catheter, and preferably, the partitioning element is located in the delivery catheter, and preferably, the partitioning element is located in the delivery catheter adjacent a distal end thereof, and preferably, the operating element extends through the delivery catheter from a proximal end of the catheter to the partitioning element, and preferably, the operating element is releasably coupled to the connector of the partitioning element with the partitioning element adjacent the distal end of the delivery catheter.

In one embodiment of the invention the operating element is configured for drawing the partitioning element from the lumen into the delivery element through the distal end thereof, and preferably, the operating element is engageable with the connector of the partitioning element for urging the partitioning element into the delivery element. Advantageously, the delivery element cooperates with the one of the isolating element and the at least two lanyards for reducing the transverse cross-sectional area of the carrier element adjacent the proximal end of the carrier element as the one of the isolating element and the at least two lanyards are being urged into the delivery element through the distal end thereof.

In another embodiment of the invention a pair of the partitioning elements are delivered through the lumen to substantially sealably isolate the first portion of the lumen from the second portion thereof and from a third portion of the lumen remote from the second portion thereof, a first one of the partitioning elements being located in the lumen for substantially sealably isolating the first portion of the lumen from the second portion thereof, and the second one of the partitioning elements being located in the lumen for isolating the first portion thereof from the third portion thereof. Preferably, the two partitioning elements are sequentially placed in the lumen by the delivery means. Advantageously, the partitioning element located in the lumen between the first and third portions thereof on the distal end of the first portion is delivered to the lumen prior to the partitioning element located in the lumen between the first portion of the lumen and the second portion thereof on the proximal end of the first portion, and preferably, the first and second partitioning elements are spaced apart from each other.

The invention also provides a method for insufflating a first portion of a lumen in which a minimally invasive procedure is to be carried out without insufflating an adjacent second portion of the lumen, the method comprising:
providing a partitioning element expandable radially from a first state defining a first transverse cross-sectional area to a second state defining a second transverse cross-sectional area greater than the first transverse cross-sectional area,
placing the partitioning element in the first state thereof in the lumen between the first portion thereof and the second portion thereof,
radially expanding the partitioning element from the first state thereof until the partitioning element engages an inner surface of the lumen with the partitioning element extending transversely across the lumen substantially sealably isolating the first portion thereof from the second portion thereof, and
insufflating the first portion of the lumen by an insufflating gas.

Preferably, the partitioning element comprises:
an expandable carrier element extending from a proximal end to a distal end, and having a bore extending longitudinally therethrough and being radially expandable from a first state defining the first transverse cross-sectional area to the second state defining the second transverse cross-sectional area,
an isolating element carried on and secured to the carrier element and being urgeable from a non-isolating state to an isolating state for substantially sealably isolating the first portion of the lumen from the second portion thereof, the isolating element being urgeable from the non-isolating state to the isolating state as the carrier element expands from the first state to the second state, and
a connector formed by a part of the carrier element or by a part of the isolating element, or being connected either to the isolating element or to at least two lanyards secured to the periphery of the carrier element at circumferentially spaced apart intervals adjacent the proximal end thereof.

Advantageously, a communicating opening extends through the partitioning element, and insufflating gas is delivered to the first portion of the lumen through the communicating opening.

Preferably, a pair of the partitioning elements are provided for isolating the first portion of the lumen from the second portion thereof and from a third portion of the lumen remote from the second portion thereof without insufflating either the second or the third portion of the lumen, a first one of the partitioning elements is placed in the lumen at a location between the first and second portions thereof, and a second one of the partitioning elements is placed in the lumen at a location between the first and third portions thereof for isolating the first portion of the lumen from the third portion thereof, and advantageously, the first and second ones of the partitioning elements are sequentially placed in the lumen, and preferably, the second one of the partitioning elements is placed in the lumen before the first one of the partitioning elements is placed therein.

Additionally, the invention provides a lumen comprising a first portion thereof and a second portion thereof substantially sealably isolated from the first portion thereof by the partitioning element according to the invention.

In another embodiment of the invention the first portion of the lumen is isolated from a third portion of the lumen remote of and distally from the second portion of the lumen by another one of the partitioning elements according to the invention.

Preferably, the partitioning element isolating the first portion of the lumen from the second portion thereof comprises the isolating element having the communicating opening extending therethrough, and advantageously, the partitioning element isolating the first portion of the lumen from the third portion thereof is provided with the isolating element without the communicating opening extending therethrough.

Additionally, the invention provides a lumen having a first portion thereof substantially sealably isolated from a second portion of the lumen by a partitioning element according to the invention. Preferably, the partitioning element is located adjacent a proximal end of the first portion of the lumen.

In another embodiment of the invention the first portion of the lumen is substantially sealably isolated from respective second and third portions of the lumen at respective spaced apart opposite ends of the first portion thereof by a pair of the partitioning elements according to the invention, one of the partitioning elements being located adjacent the proximal end of the first portion between the first portion and the second portion of the lumen, and the other partitioning element being located adjacent a distal end of the first portion of the lumen between the first portion and the third portion of the lumen. Preferably, the partitioning element located adjacent the proximal end of the first portion of the lumen is provided with the communicating opening, and the partitioning element located adjacent the distal end of the first portion of the lumen is provided without the communicating opening.

In one embodiment of the invention the lumen comprises a lumen in the body of a human or animal subject.

In another embodiment of the invention the lumen comprises one of the duodenum, the colon and the oesophagus.

Additionally the invention provides a sealing device for securing to an instrument delivery conduit or to an instrument around the outer periphery thereof to substantially form a seal between the delivery conduit or the instrument and an internal surface of a lumen in which the delivery conduit or the instrument is located, the sealing device comprising an expandable carrier element securable to the delivery conduit or to the instrument, the carrier element being expandable from an unexpanded first state to an expanded second state, an isolating element mounted on the carrier element and urgeable by the carrier element from a non-isolating state to an isolating state to sealably engage the inner surface of the lumen as the carrier element expands from the first state thereof.

In one embodiment of the invention the carrier element comprises a self-expanding element. In another embodiment of the invention a retaining means is provided for retaining the carrier element in the first state, and preferably, the retaining means is urgeable from a retaining state retaining the carrier element in the first state, to a release state releasing the carrier element to expand from the first state to the second state.

In one embodiment of the invention the carrier element is inherently configured to expand from the first state to the second state in response to the retaining means being urged from the retaining state to the release state.

In another embodiment of the invention the carrier element comprises a memory metal, and preferably, the normal unrestrained state of the carrier element comprises the expanded second state thereof.

In one embodiment of the invention the carrier element is of tubular construction extending from a first end to a second end with a bore extending therethrough from the first end to the second end. Preferably, the first end of the carrier element comprises a non-expandable first end and is configured for securing to the delivery conduit or the instrument.

Advantageously, the second end of the carrier element comprises an expandable end and defines an outer peripheral surface. Preferably, the isolating element is secured to the carrier element around the outer peripheral surface of the carrier element adjacent the second expandable end thereof.

In one embodiment of the invention the carrier element is of lattice type construction, and in another embodiment of the invention the carrier element is machined from a tube of the memory metal.

Alternatively, the carrier element comprises a woven tube woven from the memory metal.

In one embodiment of the invention the memory metal comprises Nitinol.

In another embodiment of the invention the carrier element is of stent type construction.

In one embodiment of the invention the isolating element comprises an isolating membrane, and preferably, the isolating membrane defines an outer peripheral portion, and preferably, the outer peripheral portion of the isolating membrane is secured to the carrier element.

In another embodiment of the invention the isolating membrane defines an opening therethrough for accommodating the delivery conduit or the instrument therethrough. Advantageously, the opening extending through the isolating membrane defines an inner rim, and preferably, the isolating membrane is secured to the delivery conduit or the instrument around the inner rim defined by the opening extending therethrough.

In one embodiment of the invention the isolating membrane is sealably secured to the delivery conduit or to the instrument around the inner rim defined by the opening.

In one embodiment of the invention the isolating element and the carrier element are releasably secured to the delivery conduit or the instrument, and in an alternative embodiment of the invention the isolating element and the carrier element are non-releasably secured to the delivery conduit or to the instrument.

In one embodiment of the invention the isolating membrane comprises a flexible membrane, and in another embodiment of the invention the isolating membrane comprises an expandable material. Alternatively, the isolating membrane comprises a non-expandable material.

Preferably, the isolating membrane is foldable, and advantageously, is foldable to a collapsed state when the carrier element is in the unexpanded first state.

In another embodiment of the invention the isolating membrane is substantially impermeable to gas, and in particular, is substantially impermeable to an insufflating gas.

In one embodiment of the invention the retaining means extends at least the length of the expandable part of the carrier element, and preferably, an urging means is provided to urge the retaining means between the retaining state and the release state. Preferably, the urging means comprises a manually operable urging means.

In one embodiment of the invention the retaining means comprises an elongated retaining sleeve extendable over the carrier element, and preferably, the elongated retaining sleeve extends around the delivery conduit or the instrument, and preferably, the retaining sleeve extends to the proximal end of the delivery conduit or the instrument.

Alternatively, the retaining means comprises a retaining ring.

In one embodiment of the invention the retaining means is slideably mounted on the delivery conduit or the instrument from the retaining state to the released state.

In another embodiment of the invention the sealing device is located adjacent a distal end of the delivery conduit or the instrument.

In one embodiment of the invention the sealing device is releasably secured to the delivery conduit or the instrument, and in an alternative embodiment of the invention the sealing device is non-releasably secured to the delivery conduit or the instrument.

In one embodiment of the invention the delivery conduit comprises a delivery catheter, and in another embodiment of the invention the delivery conduit comprises an endoscope. In a further embodiment of the invention the delivery conduit is adapted for delivering insufflating gas therethrough.

In another embodiment of the invention the instrument comprises an instrument suitable for carrying out a minimally invasive procedure in the body of a human or animal subject, and in a further embodiment of the invention the instrument comprises an endoscope.

The invention also provides a method for sealably locating a delivery conduit or an instrument in a lumen, the method comprising securing the sealing device according to the invention to the delivery conduit or the instrument, and expanding the carrier element from the unexpanded first state for urging the isolating membrane into sealable engagement with the inner surface of a wall of the lumen.

In one embodiment of the invention the delivery conduit or the instrument is urged into the lumen with the retaining means retaining the carrier element in the unexpanded first state, and preferably, the retaining means is urged from the retaining state to the release state when the delivery conduit or the instrument is located at a desired location in the lumen for permitting the carrier element to expand from the unexpanded first state.

The invention also provides a delivery conduit comprising the sealing device according to the invention mounted on the delivery conduit and extending around an outer periphery thereof.

The invention also provides an instrument comprising the sealing device according to the invention mounted on the instrument and extending around an outer periphery thereof.

In one embodiment of the invention the sealing device is releasably secured to the delivery conduit or the instrument, and in another embodiment of the invention the sealing device is non-releasably secured to the delivery conduit or the instrument.

In another embodiment of the invention the sealing device is located adjacent the distal end of the delivery conduit or the instrument, and preferably, the sealing device is located spaced apart proximately from the distal end of the delivery conduit or the instrument.

### Advantages of the Invention

The advantages of the invention are many. The partitioning element according to the invention and the method according to the invention provide for the efficient isolation of, for example, a first portion of an elongated lumen, such as the duodenum, from a second portion thereof in order to permit insufflating of the first portion of the lumen with minimal or no leakage of insufflating gas from the insufflated first portion to the uninsufflated second portion of the lumen. This, therefore enables a surgeon or clinician to insufflate a part of an elongated lumen in which a minimally invasive investigative or a surgical procedure is to be carried out without having to insufflate the lumen over its entire length. Accordingly, by limiting the portion of the lumen to be insufflated to that portion, in which the procedure is to be carried out, any post operative stress or pain which may be suffered by a subject is minimised, and in many cases, is avoided.

By utilising two of the partitioning elements in an elongated lumen with the partitioning elements spaced apart to define a first portion of the lumen in which an investigative or surgical procedure is to be carried out, and which is to be insufflated, only that first portion of the lumen at which the procedure is to be carried out is insufflated with minimal or no leakage of insufflating gas from the first portion being insufflated to either one of a proximal uninsufflated second portion of the lumen or a distal uninsufflated third portion of the lumen. This, further, minimises and in general avoids any post operative stress or pain, which may otherwise be suffered by a subject.

Another important advantage of the invention is that the carrier element of each partitioning element, once expanded from the first unexpanded state results in the partitioning element substantially sealably isolating the first portion of the lumen from the adjacent second or third portions thereof, and the carrier element remains in the expanded state without support from any external support systems. Accordingly, where two spaced apart partitioning elements are located in an elongated lumen to isolate a first portion of the lumen between the two partitioning elements from the remaining second and third proximal and distal portions of the lumen, there is no need for any tubes, wires or other connecting systems for connecting the partitioning elements to an external support system, for example, to a source of pressurised air or gas, which might otherwise be required for maintaining the partitioning elements in the expanded state substantially sealably engaging the inner surface of the lumen. This is a particularly important advantage, in that it leaves the first portion of the lumen between the two spaced apart partitioning elements in which the investigative or surgical procedure is to be carried out free of any trailing connecting systems, such as tubes, wires or other connecting and support systems.

A further advantage of the invention is that the partitioning elements are particularly suitable for temporarily sealably isolating a portion of a lumen to be insufflated, and in which a minimally invasive procedure is to be carried out, from an adjacent portion of the lumen or from the remainder of the lumen, and are easily deployed in the lumen and are also easily removed from the lumen.

A further advantage of the invention is achieved by providing the isolating membrane at the proximal end of the carrier elements. This enables the partitioning elements to be readily easily withdrawn into and captured in the delivery element during removal of the partitioning element from the lumen on completion of the procedure. By virtue of the connector being connected to the outer periphery of the carrier element through the isolating membrane adjacent the proximal end thereof, and the connector being located centrally relative to the carrier element, when the connector is being pulled proximally by the operating element, the pulling force on the connector is evenly distributed around the outer periphery of the carrier element. Therefore, while the distal end of the carrier element remains engaged with the lumen and stationary therein, the continuous pulling of the connector by the operating element proximally results in lengthening of the carrier element, which in turn results in the diameter of the carrier element adjacent the proximal end thereof reducing. Progressive pulling on the connector proximally results in further reduction of the diameter of the proximal end of the carrier element thereby facilitating entry of the proximal end of the carrier element into the delivery element for removal of the partitioning element from the lumen. Similarly, the provision of the lanyards connecting the connector to the proximal end of the carrier element of the partitioning elements also achieves a similar advantage. A further advantage of providing the lanyards connecting the connector to the proximal end of the carrier element prevents overstressing of the isolating element during pushing or pulling on the connector.

The invention will be more clearly understood from the following description of some preferred embodiments thereof, which are given by way of example only with reference to the accompanying drawings.

### BRIEF DESCRIPTION OF THE DRAWINGS

FIG. 1 is a cross-sectional side elevational view of a portion of a lumen according to the invention, in this case a duodenum comprising a pair of partitioning elements, also according to the invention sealably isolating a first portion of the duodenum from respective proximal second and distal third portions thereof,

FIG. 2 is a side elevational view of one of the partitioning elements of FIG. 1,

FIG. 3 is an end elevational view of the partitioning element of FIG. 2,

FIG. 4 is a cross-sectional side elevational view of the partitioning element of FIG. 2,

FIG. 5 is a cross-sectional side elevational view of the partitioning element of FIG. 2 in a different state to that of FIG. 2,

FIG. 6 is a side elevational view of another one of the other one of the partitioning elements of FIG. 1,

FIG. 7 is an end elevational view of the partitioning element of FIG. 6,

FIG. 8 is a cross-sectional side elevational view of the partitioning element of FIG. 6,

FIG. 9 is a cross-sectional side elevational view of a delivery catheter also according to the invention comprising one of the partitioning elements of FIG. 1 in the state of FIG. 5,

FIG. 10 is a cross-sectional side elevational view of the lumen of FIG. 1 illustrating one of the partitioning elements of FIG. 1 being positioned in the lumen by the delivery catheter of FIG. 9,

FIG. 11 is a cross-sectional side elevational view of the lumen of FIG. 1 illustrating one of the partitioning elements of FIG. 1 being removed from the lumen by the delivery catheter of FIG. 9,

FIG. 12 is a cross-sectional side elevational view of the lumen of FIG. 1 illustrating one of the partitioning elements of FIG. 1 being positioned in the lumen by the delivery catheter of FIG. 9 through an endoscope,

FIG. 13 is a side elevational view of a partitioning element according to another embodiment of the invention,

FIG. 14 is an end elevational view of the partitioning element of FIG. 13,

FIG. 15 is a cross-sectional side elevational view of the partitioning element of FIG. 13,

FIG. 16 is a side elevational view of a partitioning element according to another embodiment of the invention,

FIG. 17 is an end elevational view of the partitioning element of FIG. 16,

FIG. 18 is a cross-sectional side elevational view of the partitioning element of FIG. 16,

FIG. 19 is a cross-sectional side elevational view of a lumen according to another embodiment of the invention comprising a pair of partitioning elements sealably isolating a first portion of the lumen from a proximal second portion and a distal third portion of the lumen,

FIG. 20 is a side elevational view of one of the partitioning elements of FIG. 19,

FIG. 21 is an end elevational view of the partitioning element of FIG. 20,

FIG. 22 is an end elevational view of the partitioning element of FIG. 20 from the opposite end to that of FIG. 21,

FIG. 23 is a cross-sectional side elevational view of the partitioning element of FIG. 20,

FIG. 24 is a cross-sectional side elevational view of a delivery catheter according to another embodiment of the invention comprising one of the partitioning elements of FIG. 19 in a different state to that of FIG. 19,

FIG. 25 is a side elevational view of the other one of the partitioning elements of FIG. 19,

FIG. 26 is an end elevational view of the partitioning element of FIG. 25,

FIG. 27 is an end elevational view of the partitioning element of FIG. 25 from the opposite end to that of FIG. 26,

FIG. 28 is a cross-sectional side elevational view of the partitioning element of FIG. 25,

FIG. 29 is a side elevational view of a partitioning element according to another embodiment of the invention,

FIG. 30 is an end elevational view of the partitioning element of FIG. 29,

FIG. 31 is an end elevational view of the partitioning element of FIG. 29 from the opposite end to that of FIG. 30,

FIG. 32 is a cross-sectional side elevational view of the partitioning element of FIG. 29 on the line XXXII-XXXII of FIG. 30,

FIG. 33 is a cross-sectional end elevational view of the partitioning element of FIG. 29 on the line XXXIII-XXXIII of FIG. 29,

FIG. 34 is a side elevational view of the partitioning element of FIG. 29 in a different state to that of FIG. 29,

FIG. 35 is a cross-sectional side elevational view of the partitioning element of FIG. 29 in the state of FIG. 34,

FIG. 36 is a side elevational view of the partitioning element of FIG. 29 releasably secured to an operating element,

FIG. 37 is a cross-sectional side elevational view of a partitioning element according to another embodiment of the invention,

FIG. 38 is a side elevational view of a delivery conduit according to the invention comprising a sealing device also according to the invention,

FIG. 39 is an end elevational view of the delivery conduit of FIG. 38,

FIG. 40 is a cross-sectional side elevational view of the delivery conduit of FIG. 38,

FIG. 41 is a partly cross-sectional side elevational view of the delivery conduit of FIG. 38 illustrating the sealing device in a different state to that of FIG. 38,

FIG. 42 is a cross-sectional side elevational view of a lumen comprising the delivery conduit of FIG. 38 located therein, and

FIG. 43 is a cross-sectional side elevational view of a lumen comprising the partitioning element of FIG. 25 and the delivery conduit of FIG. 38 sealably isolating a first portion of the lumen from a proximal second portion and from a distal third portion thereof.

### DETAILED DESCRIPTION OF PREFERRED EMBODIMENTS OF THE INVENTION

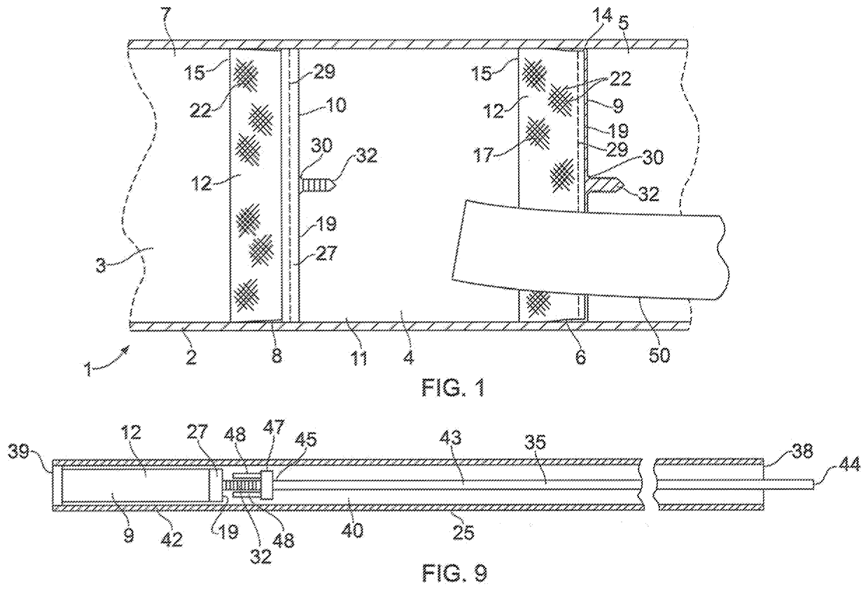

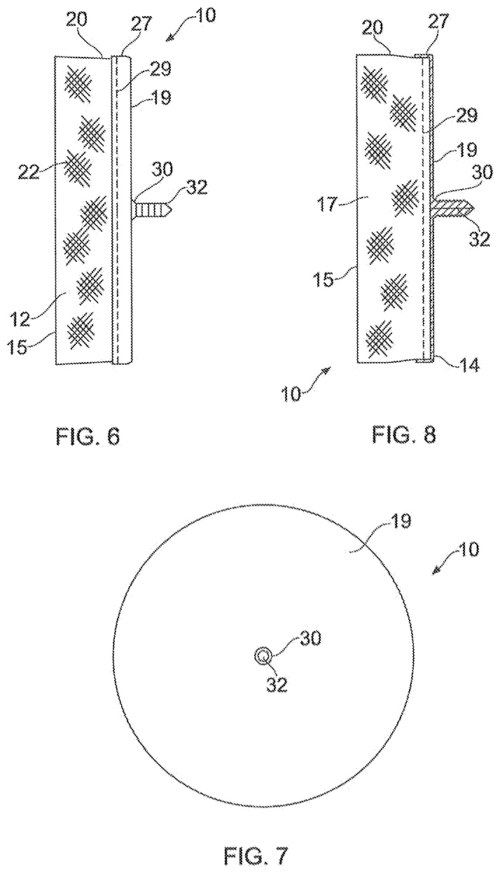

Referring to the drawings, and initially to FIGS. 1 to 12 thereof, there is illustrated an elongated lumen according to the invention and indicated generally by the reference numeral 1, which in this embodiment of the invention comprises a duodenum 2 of a human subject. The duodenum 2 defines a passageway 3 extending longitudinally therethrough comprising a first portion 4, in which a minimally invasive procedure is to be carried out endoscopically, and which is to be insufflated without insufflating the remainder of the duodenum 2. The procedure may be an investigative or a surgical or other procedure, as will be well known to those skilled in the art. The first portion 4 of the passageway 3 of the duodenum 2 is substantially isolated from a proximal second portion 5 thereof and from a distal third portion 7 thereof by respective partitioning elements, namely, a first partitioning element according to the invention and indicated generally by the reference numeral 9, and a second partitioning element also according to the invention and indicated generally by the reference numeral 10. Although only a small portion of each of the first and second portions 5 and 7 of the duodenum 2 are illustrated, the second portion 5 represents the portion of the duodenum 2 extending proximally from the first portion 4 thereof to the corresponding end (not shown) of the duodenum 2, and the third portion 7 represents the portion of the duodenum 2 extending distally from the first portion 4 thereof to the corresponding other end (also not shown) of the duodenum 2.

The first partitioning element 9 is located in the passageway 3 of the duodenum 2 at a first location 6 between the first portion 4 of the duodenum 2 and the second portion 5 thereof, and substantially sealably engages an inner surface 11 of the duodenum 2 adjacent the first location 6 for substantially sealably isolating the first portion 4 from the second portion 5. The second partitioning element 10 is located in the passageway 3 spaced apart distally from the first partitioning element 9 at a second location 8 between the first portion 4 of the duodenum and the third portion 7 thereof, and substantially sealably engages the inner surface 11 of the duodenum 2 adjacent the second location 8 for substantially sealably isolating the first portion 4 from the third portion 7. Therefore, when the first portion 4 is substantially sealably isolated from the second and third portions 5 and 7 of the passageway 4 of the duodenum 2 by the first and second partitioning elements 9 and 10, respectively, the first portion 4 may be insufflated with a suitable insufflating gas with minimal leakage of the insufflating gas from the first portion 4 to the second and third portions 5 and 7.

The first and second partitioning elements 9 and 10 are placed in the duodenum 2 at the first and second locations 6 and 8 by a delivery catheter 25, which will be described below, and are also withdrawn from the duodenum by the delivery catheter 25, as will also be described below.

The first and second partitioning elements 9 and 10 are substantially similar to each other, and the first partitioning element 9 will first be described.

The first partitioning element 9 comprises an elongated expandable carrier element, in this embodiment of the invention a radially self-expanding carrier element 12 of tubular construction extending from a proximal end 14 to a distal end 15, and having a bore 17 extending centrally therethrough from the proximal end 14 to the distal end 15. An isolating element, in this embodiment of the invention comprising an isolating membrane 19 is secured to the carrier element 12 around an outer peripheral surface 20 thereof adjacent the proximal end 14 of the carrier element 12 to close the proximal end 14 thereof. Thus, when the carrier element 12 expands radially in the passageway 3 of the duodenum 2 and engages the inner surface 11 of the duodenum 2, the isolating membrane 19 extends transversely across the passageway 3 of the duodenum 2 and substantially sealably engages the inner surface 11 of the duodenum 2 to sealably isolate the first portion 4 of the passageway 3 from the proximal second portion 5 thereof.

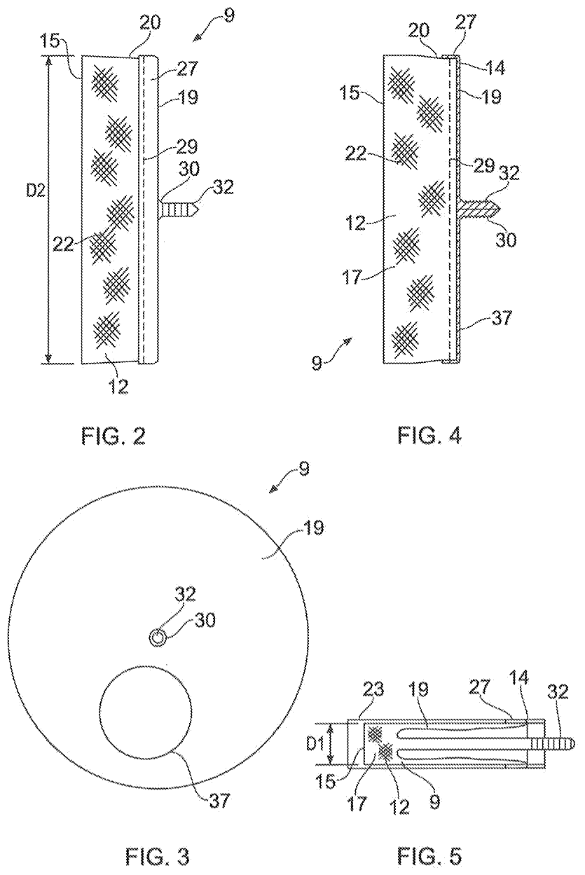

The carrier element 12 is of stent like construction, and is formed by a plurality of strands of a memory metal, in this embodiment of the invention a plurality of wires 22 of Nitinol. The wires 22 of the memory metal extend generally helically around or partly around the carrier element from the proximal end 14 to the distal end 15, and are woven to form the carrier element 12 in a manner similar to that used in the formation of a stent and will be understood by those skilled in the art. The carrier element 12 is of cylindrical shape, and is radially expandable from an unexpanded first state defining a circular first transverse cross-sectional area of diameter D1 illustrated in FIG. 5 to an expanded second state defining a circular second transverse cross-sectional area of diameter D2 illustrated in FIGS. 2 to 4. The second transverse cross-sectional area is greater than the first transverse cross-sectional area.

The carrier element 12 during the manufacture thereof, is temperature set in the expanded second state defining the second transverse cross-sectional area, and is subsequently compressed to the unexpanded first state defining the first transverse cross-sectional area. In the unexpanded first state, the carrier element 12 is unstable, and is retained in the first state in a tubular cartridge 23. The second transverse cross-sectional area defined by the carrier element 12 in the second state thereof is selected to be greater than the diameter of the passageway 3 extending through the duodenum 2 adjacent the location 6, so that the carrier element 12, when expanded from the first state thereof, tightly engages the inner surface 11 of the duodenum 2.

Turning now to the isolating membrane 19, the isolating membrane 19 is formed from a non-resilient and non-elastic flexible foldable sheet of a suitable material, which is impermeable or substantially impermeable to insufflating gases, such as, air, carbon dioxide and the like. In this embodiment of the invention the isolating membrane 19 comprises polyurethane sheet material, although it will be readily apparent to those skilled in the art that the isolating membrane 19 may be of any other suitable sheet material, which is impermeable, substantially impermeable or of a low permeability to the specific insufflating gas to be used to insufflate the first portion 4 of the duodenum 2. During manufacture of the first partitioning element 9, the isolating membrane 19 is secured to the proximal end 14 of the carrier element 12 when the carrier element 12 is in the expanded second state. An outer circumferential peripheral portion 27 of the isolating membrane 19 is folded around the proximal end 14 of the carrier element 12, and is secured to the outer peripheral surface 20 of the carrier element 12 adjacent the proximal end 14 thereof by circumferentially suturing the outer peripheral portion 27 to the carrier element 12 by sutures 29. Accordingly, as the carrier element 12 expands from the unexpanded first state to the expanded second state, the isolating membrane is urged from a non-isolating state illustrated in FIG. 5 to an isolating state illustrated in FIGS. 2 to 4, so that when the first partitioning element 9 is located at the first location 6 in the duodenum 2 with the isolating membrane 19 in the isolating state, the first portion 4 of the duodenum 2 is substantially sealably isolated from the second portion 5 of the duodenum 2.

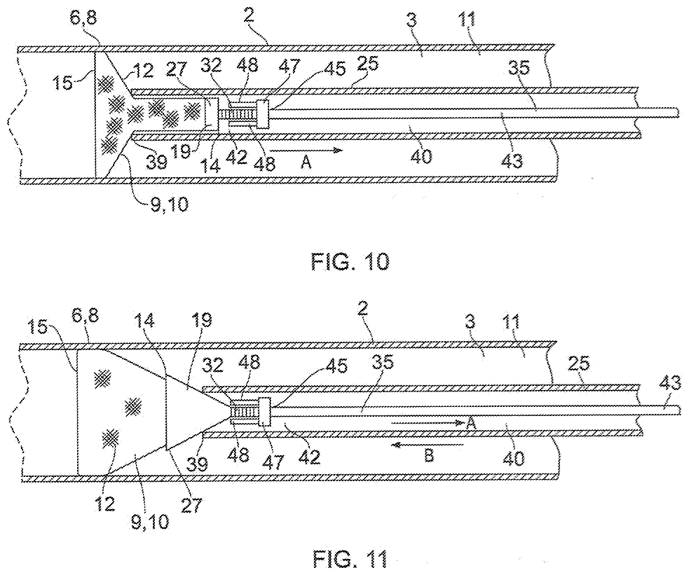

The area of the isolating membrane 19 is sufficiently large to permit a central portion 30 of the isolating membrane 19 to be gathered together as clearly illustrated in FIGS. 2 to 5 when the carrier element 12 is in the expanded second state thereof. The gathered central portion 30 forms a connector, in this case, a connecting tab 32 which is adapted to be engaged by an operating element 35 which cooperates with the delivery catheter 25, as will be described below with reference to FIGS. 10 to 12, for deploying and withdrawing the first partitioning element 9 at and from the first location 6. The connecting tab 32 is formed by securing the folds of the central portion 30 of the isolating membrane 19 together by wire wrapping, or by any other suitable means, for example, by bonding the folds of the central portion 30 together by a suitable adhesive, by heat welding the folds of the central portion 30 together, or by any other suitable means.

A communicating opening 37 extends through the isolating membrane 19 for accommodating an instrument or an endoscope through the isolating membrane 19, and in turn through the bore 17 of the carrier element 12 of the first partitioning element 9 into the first portion 4 of the passageway 3 of the duodenum 2. The communicating opening 37 is dimensioned to form a reasonably tight fit on the instrument or endoscope which is to be passed therethrough into the first portion 4 of the passageway 3 of the duodenum 2 in order to maintain a substantially airtight seal between the isolating membrane 19 and the instrument or the endoscope around the instrument or the endoscope. An endoscope 50 is illustrated in FIG. 1 extending into the first portion 4 of the duodenum 2 through the communicating opening 37 in the isolating membrane 19 of the first partitioning element 9.

It is also envisaged that one or more additional communicating openings, which would typically be of diameter less than the diameter of the communicating opening 37, may be formed in the isolating membrane 19 to accommodate one or more tubular members therethrough into the first portion 4 for accommodating insufflating gas therethrough for insufflating the first portion 4 of the passageway 3, or for accommodating an instrument therethrough into the first portion 4. Such a tubular member is disclosed in PCT Published Application No. WO 2021/209983 of the present applicant, and may or may not be releasably secured, for example, to an endoscope by clips disclosed in PCT Published Application No. WO 2021/209983.

As discussed above, the isolating membrane 19 is secured to the carrier element 12 while the carrier element 12 is in the expanded second state. Once the isolating membrane 19 has been secured to the carrier element 12, the carrier element 12 is compressed from the second expanded state to the first unexpanded state. As the carrier element 12 is being compressed from the second state to the first state, the isolating membrane 19 is folded to lie within the bore 17 extending through the carrier element 12 with the connecting tab 32 extending proximally from the proximal end 14 of the carrier element 12 to facilitate gripping of the tab 32 by the operating element 35. The compressed carrier element 12 in the unexpanded first state with the isolating membrane folded within the carrier element 12 is inserted into the cartridge 23, and is retained therein in the first unexpanded state until the first partitioning element 9 is to be used.

Turning now to the second partitioning element 10, the second partitioning element 10 is substantially similar to the first partitioning element 9, and similar components are identified by the same reference numerals. The only difference between the second partitioning element 10 and the first partitioning element 9 is that the communicating opening 37 formed in the isolating membrane 19 of the first partitioning element 9 is omitted from the isolating membrane 19 of the second partitioning element 10. The communicating opening 37 is not required in the second partitioning element 10, since in general, there will be no requirement to enter an endoscope, instrument or the like into the first portion 4 of the duodenum 2 through the second partitioning element 10 from the distal third portion 7 of the duodenum 2. However, if there was such a requirement, the second partitioning element 10 would also be provided with a communicating opening extending through the isolating membrane, similar to the communicating opening 37 of the first partitioning element 9, which may or may not be appropriately sized to sealably engage the endoscope or the instrument passing therethrough.

Turning now to the delivery catheter 25 and the operating element 35, and referring now to FIGS. 9 to 12, the delivery catheter 25 comprises an elongated bendable catheter extending from a proximal end 38 to a distal end 39. An elongated bore 40 extends longitudinally through the delivery catheter 25 from the proximal end 38 to the distal end 39. A distal portion 42 of the catheter bore 40 extending inwardly from the distal end 39 thereof is of a diameter just greater than the outside diameter of the first transverse cross-sectional area of the carrier element 12 when the carrier element 12 is in the unexpanded first state thereof for facilitating insertion of each one of the first and second partitioning elements 9 and 10 into the distal portion 42 of the bore 40 of the delivery catheter 25 from the cartridge 23. The partitioning element 9 or 10 is inserted into the distal portion 42 of the bore 40 from the distal end 39 thereof with the proximal end 14 of the carrier element 12 leading, and the one of the first and second partitioning elements 9 or 10 is urged into the distal portion 42 of the catheter bore 40 until the distal end 15 of the carrier element 12 is just inside the distal end 39 of the distal portion 42 of the delivery catheter 25, see FIG. 9.

The operating element 35 comprises an elongated operating member 43 of bendable material, which is bendable with the delivery catheter 25, and is slidable in the bore 40 extending through the delivery catheter 25. The operating member 43 extends from a proximal end 44 to a distal end 45. The proximal end 44 of the operating member 43 extends proximally outwardly from the bore 40 of the delivery catheter 25 adjacent the proximal end 38 thereof. The distal end 45 of the operating member 43 terminates in an engagement means configured to releasably engage the connecting tab 32 of the isolating membrane 19 of the one of the first and second partitioning elements 9 or 10 in the distal portion 42 of the delivery catheter 25, see FIG. 9. Any suitable engagement means for engaging the connecting tab 32 may be provided adjacent the distal end 45 of the operating member 43. In this embodiment of the invention the distal end 45 of the operating member 43 terminates in a gripper element 47 comprising a pair of jaws 48 which are spring urged together to tightly engage the connecting tab 32 therebetween. The jaws 48 are operated between a closed state and an open state by a hand operated lever (not shown) located adjacent the proximal end of the operating member 43 through an operating wire (also not shown). The operating wire (not shown) extends through an internal bore (not shown) extending longitudinally through the operating member 43 or extends along the outer side of the operating member 43 from the hand operated lever to the gripper element 47.

The gripper element 47 of the operating element 35 is configured to grip the connecting tab 32 of the relevant partitioning element 9 or 10 located in the distal portion 42 of the bore 40 of the delivery catheter 25, so that when the distal end 39 of the delivery catheter 25 is accurately positioned at the location 6 or 8, at which the relevant partitioning element 9 or 10 is to be located in the passageway 3 of the duodenum 2, the operating element 35 holds the partitioning element in position relative to the duodenum 2. Thus, with the partitioning element 9 or 10 still in the distal portion 42 of the bore 40 of the delivery catheter 25 and correctly located in the duodenum 2, and held in position by the operating element 35, the delivery catheter 25 may be withdrawn proximally in the direction of the arrow A relative to the partitioning element 9 or 10 and the operating element 35, see FIG. 10, so that on completion of withdrawal of the delivery catheter 25 from the relevant one of the partitioning elements 9 or 10, the partitioning element 9 or 10 is discharged from the delivery catheter 25. The partitioning element 9 or 10 on being discharged from, and being no longer restrained in the unexpanded first state by the distal portion 42 of the delivery catheter 25, expands under its own inherent resilience from the first state thereof towards the expanded second state thereof until the carrier element 12 and the outer peripheral portion 27 of the isolating membrane 19 are tightly abutting the inner surface 11 of the duodenum 2, and the isolating membrane 19 is in the isolating state. With the carrier element 12 and the isolating membrane 19 of the partitioning element 9 or 10 tightly abutting the duodenum 2, the isolating membrane 19 is in the isolating state extending transversely across the passageway 3 of the duodenum 2 with the first portion 4 of the duodenum 2 substantially sealably isolated from the corresponding one of the second portion 5 or the third portion 7, depending on the partitioning element 9 or 10 being deployed in the duodenum 2.

During discharge of the partitioning element 9 or 10 from the distal portion 42 of the bore 40 of the delivery catheter 25, due to the resistance of the carrier element 12 bearing on the inner surface of the delivery catheter 25, the operating element 35 may tend to urge the isolating membrane 19 and the connecting tab 32 further into the bore 17 of the carrier element 12. However, the isolating membrane 19 is of sufficient strength to withstand any urging of the isolating membrane 19 further inwardly into the bore 17 of the carrier element 12.

The delivery catheter 25 and the operating element 35 are also used to withdraw the partitioning elements 9 and 10 from the duodenum 2, see FIG. 11. The delivery catheter 25 with the operating element 35 located in the bore 40 thereof is urged distally into the duodenum 2 until the distal end 39 of the delivery catheter 25 is located proximally of the location 6 or 8, at which the partitioning element 9 or 10 to be withdrawn, is located. The gripper element 47 of the operating element 35 is urged distally from the distal end 39 of the delivery catheter 25 to grip the connecting tab 32. With the gripper element 47 gripping the connecting tab 32, the operating element 35 is urged in the proximal direction, namely, in the direction of the arrow A into the delivery catheter 25 for urging the connecting tab 32 into the distal portion 42 of the delivery catheter 25 through the distal end 39 thereof. The urging action of the operating element 35 pulling the connecting tab 32 of the partitioning element 9 or 10 into the distal portion 42 of the delivery catheter 25, results in the isolating membrane 19 being drawn into the distal portion 42 of the delivery catheter 25.

Further urging of the isolating membrane 19 into the distal portion 42 of the delivery catheter 25, while the distal end 15 of the carrier element 12 is engaging the duodenum 2, results in longitudinal stretching of the carrier element 12, which in turn results in a reduction in the transverse cross-sectional area of the proximal end 14 of the carrier element 12, see FIG. 11. This stretching of the carrier element 12 from the proximal end 14 thereof results in progressive reduction in the transverse cross-sectional area of the carrier element 12 in a proximal direction along the carrier element 12 towards the proximal end 14 thereof. The longitudinal stretching of the carrier element 12 results from the fact that the transverse cross-sectional area of the carrier element 12 adjacent the distal end 15 thereof will be last to reduce, and therefore the distal end 15 of the carrier element 12 will be last to disengage the duodenum 2. The more the operating element 35 pulls the connecting tab 32 into the bore 40 of the delivery catheter 25, the greater will be the lengthening of the carrier element 12, and thus, the greater the reduction in the transverse cross-sectional area adjacent the proximal end 14 of the carrier element 12. The reduction in diameter of the proximal end 14 of the carrier element 12 facilitates ease of drawing of the proximal end 14 of the carrier element 12 into the distal portion 42 of the bore 40 of the delivery catheter 25 through the distal end 39 thereof.

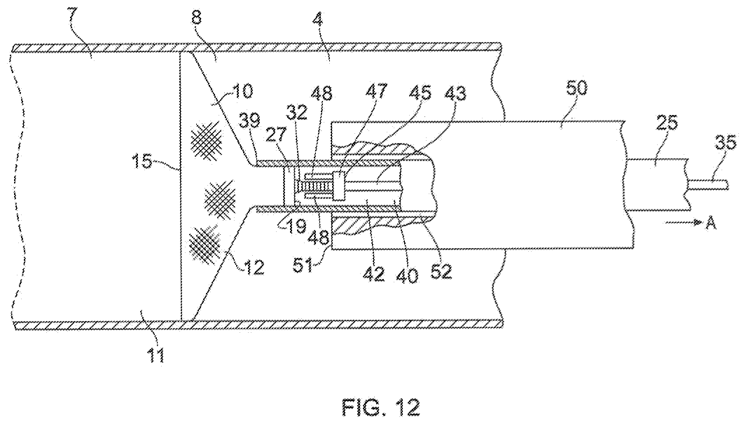

Once the connecting tab 32 and a portion of the isolating membrane 19 have been drawn into the distal portion 42 of the delivery catheter 25, the delivery catheter 25 may be urged distally in the direction of the arrow B in the duodenum 2, see FIG. 12, while the operating element 35 continues to be urged proximally in the direction of the arrow A in the bore 40 of the delivery catheter 25 to pull the connecting tab 32 proximally in the bore 40 for capturing the partitioning element 9 or 10, as the case may be, in the distal portion 42 of the bore 40 of the delivery catheter 25. This minimises proximal movement of the distal end 15 of the carrier element 12 relative to the duodenum 2 as the partitioning element 9 or 10 is being drawn into the distal portion 42 of the delivery catheter 25.

In use, the first and second partitioning elements 9 and 10 are sequentially located in the duodenum 2. The distal second partitioning element 10 is first placed and positioned at the second location 8 in the passageway 3 of the duodenum 2, and is expanded to sealably engage the duodenum 2. On placing, positioning and expanding the distal second partitioning element 10 at the second location 8 in the duodenum 2, the proximal first partitioning element 9 is placed, positioned and expanded at the first location 6 in the passageway 3 of the duodenum 2. Both the first and second partitioning elements 9 and 10 are sequentially placed in the first and second locations 6 and 8, respectively, by the delivery catheter 25 and the operating element 35 as described above with reference to FIG. 11. While the first and second partitioning elements may be placed, positioned and expanded in the duodenum 2 by the delivery catheter 25 directly entered into the duodenum, in general, the delivery catheter 25 is entered into the duodenum 2 through the endoscope 50 as illustrated in FIG. 12.

Referring now to FIG. 12, the endoscope 50 is entered into the alimentary passageway either orally or rectally, but typically orally. Initially the endoscope 50 is urged orally into the duodenum 2 and through the proximal second portion 5 thereof and into the first portion 4 until a distal end 51 of the endoscope 50 is located in the first portion 4 proximal of the second location 8, at which the second partitioning element 10 is to be located. With the second partitioning element 10 located in the distal portion 42 of the delivery catheter 25 and the operating element 35 in the bore 40 of the delivery catheter 25, and the connecting tab 32 of the second partitioning element 10 tightly gripped by the gripper element 47, the delivery catheter 25 is urged distally through an instrument channel 52 of the endoscope 50 until the distal end 39 of the delivery catheter 25 is located in the first portion 4 of the duodenum 2. The delivery catheter 25 is then maneuvered in the duodenum 2 until the distal portion 42 of the delivery catheter 25 is positioned adjacent the second location 8 between the first and third portions 4 and 7 of the duodenum 2 at which the second partitioning element 10 is to be located.

On the distal portion 42 of the delivery catheter 25 being positioned at the second location 8, the second partitioning element 10 is held in position by the operating element 35 while the delivery catheter 25 is urged proximally in the direction of the arrow A to progressively expose the second partitioning element 10, see FIGS. 10 and 12, until the second partitioning element 10 is finally discharged from the delivery catheter 25. On discharge of the second partitioning element 10 from the delivery catheter 25, the carrier element 12 expands from the first state towards its second state until the carrier element 12 and the outer peripheral portion 27 of the isolating membrane 19 abuts the inner surface 11 of the duodenum 2. With the second partitioning element 10 sealably isolating the first portion 4 of the duodenum 2 from the distal third portion 7 thereof, the gripper element 47 is disengaged from the connecting tab 32 of the second partitioning element 10, and the operating element 35 is withdrawn into the delivery catheter 25, and the delivery catheter 25 together with the operating element 35 therein are withdrawn proximally through the instrument channel 52 of the endoscope 50.

The distal portion 42 of the delivery catheter 25 is recharged with the first partitioning element 9 with the gripper element 47 of the operating element 35 tightly engaging the connecting tab 32 thereof. The endoscope 50 is withdrawn proximally from the first portion 4 of the duodenum 2 into the second portion 5 thereof until the distal end 51 of the endoscope 50 is located proximally of the first location 6 at which the first partitioning element 9 is to be placed. The delivery catheter 25 with the first partitioning element 9 in the distal portion 42 thereof is again urged distally through the instrument channel 52 of the endoscope 50 until the distal portion 42 of the delivery catheter 25 is positioning in the first location 6 between the first portion 4 and the second portion 5 of the duodenum 2. The first partitioning element 9 is then discharged from the distal portion 42 of the delivery catheter 25 in a similar manner to that described with reference to the discharge of the second partitioning element 10 from the delivery catheter 25. The first partitioning element 9 expands from the unexpanded first state towards the second state thereof, until the first partitioning element 9 sealably isolates the first portion 4 of the duodenum 2 from the second portion 5 thereof. The gripper element 47 is disengaged from the connecting tab 32 of the first partitioning element 9 and the delivery catheter 25 together with the operating element 35 are withdrawn proximally through the instrument channel 52 of the endoscope 50.

With the first and second partitioning elements 9 and 10 sealably isolating the first portion 4 of the duodenum 2 from the second and third portions 5 and 7 thereof, the first portion 4 is ready for insufflating and the carrying out the procedure therein. The endoscope 50 is next urged distally through the communicating opening 37 in the isolating membrane 19 of the proximal first partitioning element 9 into the first portion 4 of the duodenum 2. The first portion 4 of the duodenum 2 is insufflated by insufflating gas delivered through an insufflating gas accommodating channel (not shown) of the endoscope 50. The appropriate instrument or instruments for carrying out the procedure are then urged through the instrument channel 52 of the endoscope 50 into the insufflated first portion 4 of the passageway 3 of the duodenum 2 for carrying out the procedure.

On completion of the procedure, the instrument is withdrawn through the instrument channel 52 of the endoscope 50. The endoscope 50 is withdrawn proximally into the proximal second portion 5 of the passageway 3 of the duodenum 2 through the communicating opening 37 in the isolating membrane 19 of the first partitioning element 9. The delivery catheter 25 with the operating element 35 located in the bore 40 thereof is urged through the instrument channel 52 of the endoscope 50 until the distal end 39 of the delivery catheter 25 extends through the distal end 51 of the endoscope 50 and is located towards but spaced apart proximally from the proximal end 14 of the carrier element 12 of the proximal first partitioning element 9. The operating element 35 is urged distally in the delivery catheter 25 until the gripper element 47 extends distally outwardly through the distal end 39 of the delivery catheter 25. The connecting tab 32 of the first partitioning element 9 is gripped by the gripper element 47, and the operating element 35 is urged proximally in the direction of the arrow A into the bore 40 of the delivery catheter 25, for in turn drawing the isolating membrane 19 of the first partitioning element 9 into the distal portion 42 of the bore 40 of the delivery catheter 25, see FIG. 11. As the isolating membrane 19 is being drawn into the distal portion 42 of the bore 40 of the delivery catheter 25 by the operating element 35, the delivery catheter 25 is urged distally in the duodenum 2 in the direction of the arrow B relative to the operating element 35 to thereby capture the first partitioning element 9 in the distal portion 42 of the delivery catheter 25.

The delivery catheter 25 with the first partitioning element 9 captured therein, and the operating element 35, are withdrawn proximally through the instrument channel 52 of the endoscope 50. The first partitioning element 9 is then removed from the distal portion 42, of the delivery catheter 25 by urging the first partitioning element 9 distally through the distal end 39 of the delivery catheter 25 by the operating element 35.

Alternatively, once the first partitioning element 9 has been drawn into the distal portion 42 of the delivery catheter 24 from the first location 6 in the duodenum 2, instead of withdrawing the delivery catheter 25 proximally through the endoscope 50, the delivery catheter 25 may be left in the endoscope 50, and the first partitioning element 9 would be withdrawn proximally through the bore 40 of the delivery catheter 25 by the operating element 35 and removed through the proximal end 38 of the delivery catheter 25.

The second partitioning element 10 is next removed from the duodenum 2. With the operating element 35 in the bore 40 of the delivery catheter 25, and the delivery catheter in the instrument channel 52 of the endoscope 50, the endoscope 50 is urged distally into the first portion 4 of the duodenum 2 towards the second partitioning element 10, but with the distal end 51 of the endoscope 50 spaced apart proximally from the connecting tab 32 of the second partitioning element 10. The distal end 39 of the delivery catheter 25 is urged through the distal end 51 of the endoscope 50, and the gripper 47 of the operating element 35 is urged through the distal end 39 of the delivery catheter 25. The connecting tab 32 of the second partitioning element 10 is gripped by the gripper element 47, and the second partitioning element 10 is captured in the distal portion 42 of the delivery catheter 25 as already described with reference to the second partitioning element 10. The delivery catheter is then withdrawn into the instrument channel 52 of the endoscope 50, and the endoscope 50 along with the delivery catheter are withdrawn orally from the duodenum 2.

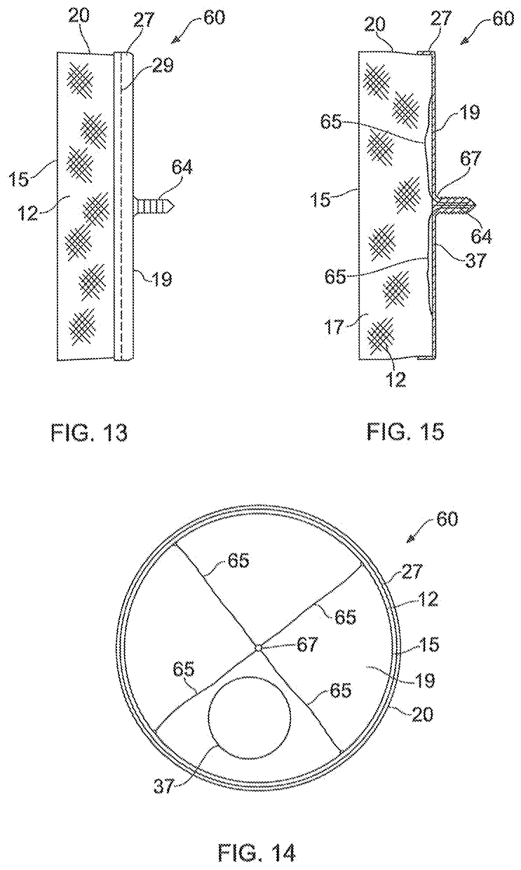

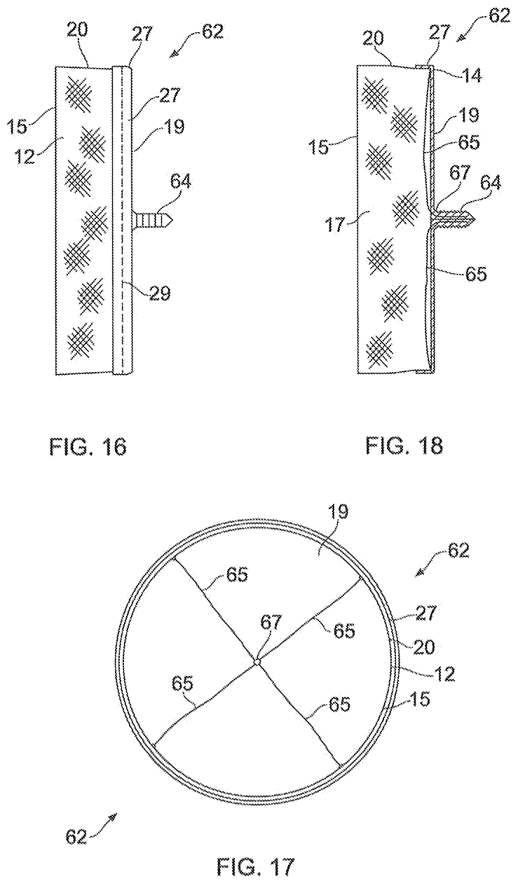

Referring now to FIGS. 13 to 18 there is illustrated first and second partitioning elements also according to the invention and indicated generally by the reference numerals 60 and 62, respectively, for also substantially sealably isolating a first portion of an elongated lumen from second and third portions thereof, for example, for substantially sealably isolating a first portion of a duodenum from respective second and third portions thereof, in a similar manner as the first and second partitioning elements 9 and 10 substantially sealably isolate the first portion 4 of the duodenum 2 from the second and third portions 5 and 7 thereof as described with reference to FIGS. 1 to 12. The first partitioning element 60 is substantially similar to the first partitioning element 9 described with reference to FIGS. 1 to 12, and similar components are identified by the same reference numerals. The second partitioning element 62 is substantially similar to the second partitioning element 10 also described with reference to FIGS. 1 to 12, and similar components are likewise identified by the same reference numerals.

The only difference between the first partitioning element 60 and the first partitioning element 9 and the second partitioning element 62 and the second partitioning element 10 is that in this embodiment of the invention the connector of each of the first and second partitioning elements 60 and 62 comprises a connecting tab 64, which is connected to the proximal end 14 of the carrier element 12 of the corresponding one of the first and second partitioning elements 60 and 62 by four lanyards formed by respective flexible connecting members 65. The flexible connecting members 65 of each one of the first and second partitioning elements 60 and 62 are secured to the outer periphery 20 of the corresponding carrier element 12 at equi-spaced apart intervals of 90° around the outer periphery 20 adjacent the proximal end 14 thereof. The flexible connecting members 65 extend from the carrier element 12 on the distal side of the isolating membrane 19 and extend to the connecting tab 64 through a central opening 67 in the corresponding isolating membrane 19, and are sealably secured in the central opening 67 of the isolating membrane 19.

In this embodiment of the invention each connecting member 65 comprises twisted fibres of nylon, polypropylene or any other suitable fibre materials, and in some embodiments of the invention may comprise twisted wire filaments. In other embodiments of the invention the connecting members 65 may be formed by extensions of four of the wires forming the corresponding carrier element 12. The length of the connecting members 65 between the periphery 20 of the carrier element 12 and the central opening 67 in the isolating membrane 19 are of sufficient length to permit unrestricted expansion of the carrier element 12 from the first state to the second state thereof.

Each connecting tab 64 is formed by the free ends of the connecting members 65 which are secured together by bonding with a suitable adhesive, fusing together by, for example, heat welding, or wire wrapping. In some embodiments of the invention the free ends of the connecting members 65 may be configured to form the connector as one or more loops.

Otherwise, the first and second partitioning elements 60 and 62 are similar to the first and second partitioning elements 9 and 10, respectively.

Use of the first and second partitioning elements 60 and 62 is similar to that described with reference to the first and second partitioning elements 9 and 10. Additionally, the deployment and withdrawal of the first and second partitioning elements 60 and 62 in and from the duodenum to isolate a first portion thereof from second and third portions thereof is likewise similar to the deployment and withdrawal of the first and second partitioning elements 9 and 10 to and from the duodenum.

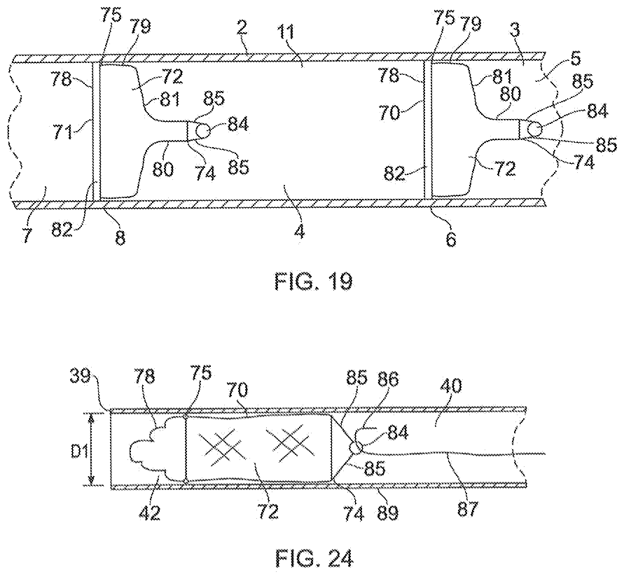

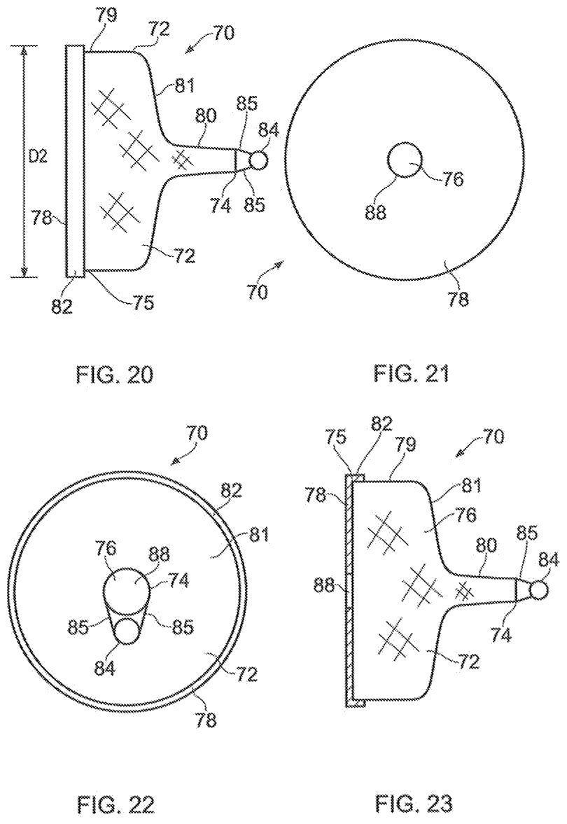

Referring now to FIGS. 19 to 28 there is illustrated an elongated lumen also according to the invention, in this case, a duodenum similar to the duodenum 2 described with reference to FIGS. 1 to 12, and similar parts of the duodenum 2 are identified by the same reference numerals, as those used in FIGS. 1 to 12. A pair of partitioning elements according to another embodiment of the invention are provided for substantially sealably isolating a first portion 4 of the duodenum 2 from second and third portions 5 and 7 thereof, respectively, in a manner substantially similar to that described with reference to FIGS. 1 to 12 whereby the partitioning elements 9 and 10 substantially sealably isolate the first portion 4 of the duodenum 2 from the second and third portions 5 and 7 thereof. A first partitioning element according to the invention of the pair of partitioning elements according to this embodiment of the invention is indicated generally by the reference numeral 70, and a second partitioning element also according to the invention of the pair of partitioning elements according to this embodiment of the invention is indicated by the reference numeral 71.

Turning initially to the first partitioning element 70, the first partitioning element 70 comprises a carrier element 72 extending from a proximal end 74 to a distal end 75 and having an elongated bore 76 extending therethrough from the proximal end 74 to the distal end 75. An isolating element comprising an isolating membrane 78 is secured to the carrier element 72 adjacent the distal end 75.

The carrier element 72 comprises an expandable carrier element which is radially self-expandable from an unexpanded first state illustrated in FIG. 24 to an expanded second state illustrated in FIGS. 20 to 23, and expands into the second state when unrestrained. The carrier element 72 is of stent construction constructed from a memory metal, for example, Nitinol, and is of a lattice type construction, and may be woven or machined from a tube of the memory metal material. The manufacture of expandable stents from memory metal will be well known to those skilled in the art. The carrier element 72 is of tubular construction of circular transverse cross-section. The carrier element 72 like the carrier elements 12 of the first and second partitioning elements 9 and 10 is temperature set in the expanded second state thereof, and is subsequently compressed into the unexpanded first state, and is retained in the unexpanded first state in a cartridge (not shown), but similar to the cartridge 23 described with reference to the first and second partitioning elements 9 and 10.

The carrier element 72 is configured to be of progressively increasing expandability from the proximal end 74 to the distal end 75, so that when the carrier element 72 expands into the second state, a distal portion 79 is formed adjacent the distal end 75, and a proximal portion 80 is formed adjacent the proximal end 74. The diameter of the bore extending through the proximal portion 80 is less than the diameter of the bore extending through the distal portion 75 in the expanded second state of the carrier element 72. However, the diameter of the bore extending through the proximal portion 80 of the carrier element 72 is sufficient to accommodate an instrument or an endoscope therethrough into the first portion of the duodenum. The distal portion 79 of the carrier element 72 transitions through an intermediate converging portion 81 to the proximal portion 80.

The isolating membrane 78 defines an outer peripheral edge portion 82 which is secured to the carrier element 72 around an outer surface of the distal portion 79 adjacent the distal end 75 thereof by any suitable securing means, for example, an adhesive, suturing or by other suitable means. In the unexpanded first state of the carrier element 72 as illustrated in FIG. 24, the isolating membrane 78 is in a non-isolating state folded within the carrier element 72, and the carrier element 72 defines a first transverse cross-sectional area of diameter D1. In the expanded second state, the carrier element 72 expands such that the distal portion 79 of the carrier element 72 defines a second transverse cross-sectional area of diameter D2 which is greater than the diameter D1 of the first transverse cross-sectional area of the carrier element 72. As the carrier element 72 expands from the unexpanded first state to the expanded second state, the isolating membrane 78 is urged from the non-isolating state illustrated in FIG. 24 to the isolating state illustrated in FIGS. 20 to 23. The diameter D2 of the second transverse cross-sectional area of the outer peripheral portion of the carrier element 72 and the isolating membrane 78 is such that the isolating membrane 78 in the isolating state thereof extends transversely across the duodenum 2 and substantially sealably engages the inner surface 11 of the duodenum 2, see FIG. 19, so that when the first partitioning element 70 is located in the first location 6 of the duodenum 2, the first portion 4 of the duodenum 2 is substantially sealably isolated from the second portion 5 thereof.

A communicating opening 88 is located centrally in the isolating membrane 78 and extends therethrough, and is centrally aligned with the portion of the bore 76 extending through the proximal portion 80 of the carrier element 72 for accommodating an endoscope or an instrument therethrough into the first portion 4 of the duodenum 2, in a similar manner as the communicating opening 37 through the isolating membrane 19 of the first partitioning element 9 likewise accommodates an endoscope or an instrument therethrough into the first portion 4 of the duodenum 2. The diameter of the communicating opening 88 is such that the isolating membrane 78 substantially sealably engage an instrument or an endoscope extending through the communicating opening 88.

A connector, in this embodiment of the invention an eye ring 84 is connected to the proximal portion 80 of the carrier element 12 adjacent the proximal end 74 thereof by a pair of wires 85. The eye ring 84 is releasably engageable by a hook 86 of an elongated operating element 87. The operating element 87 cooperates with a delivery catheter 89, similar to the delivery catheter 25 described with reference to the partitioning elements 9 and 10 for positioning and discharging the first partitioning element 70 in the duodenum 2, and also for removing the first partitioning element 70 from the duodenum 2, as will be described below. Since the delivery catheter 89 is similar to the delivery catheter 25, similar components are identified by the same reference numerals.

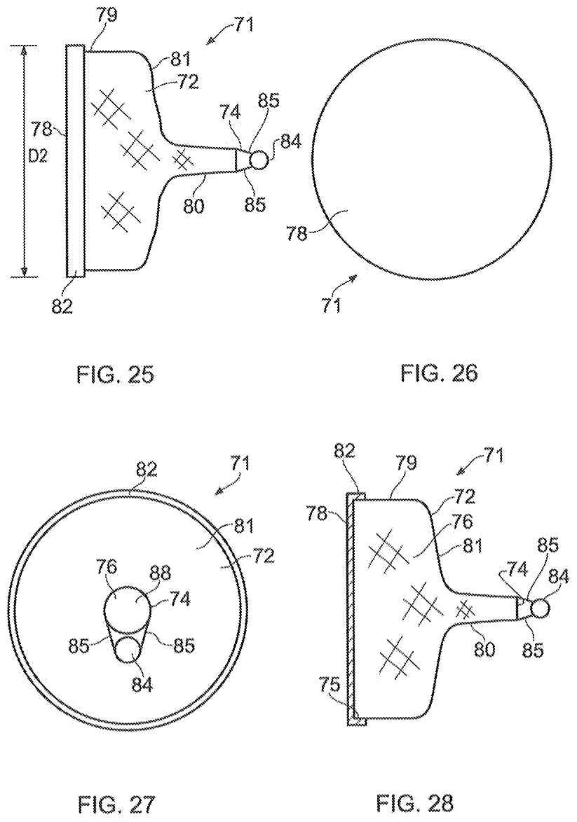

Turning now to the distal second partitioning element 71, and referring to FIGS. 25 to 28, the second partitioning element 71 is substantially similar to the first partitioning element 70, and similar components are identified by the same reference numerals. The only difference between the second partitioning element 71 and the first partitioning element 70 is that a communicating opening similar to the communicating opening 88 is omitted from the isolating membrane 78.

Otherwise, the proximal first partitioning element 70 is similar to the distal second partitioning element 71.

The use of the first and second partitioning elements 70 and 71 for sealably isolating the first portion 4 of the duodenum 2 from the second and third portions 5 and 7 thereof, is substantially similar to the use of the first and second partitioning elements 9 and 10 described with reference to FIGS. 1 to 12 for similarly isolating the first portion 4 of the duodenum 2 from the second and third portions 5 and 7 thereof. The distal second partitioning element 71 is placed and expanded in the duodenum 2 at the second location 8 between the first and third portions 4 and 7 of the duodenum 2 prior to the placing and expanding of the proximal first partitioning element 70 in the duodenum 2 at the first location 6 between the first and second portions 4 and 5 of the duodenum 2. To place the second partitioning element 71 in the duodenum 2, the second partitioning element 71 is transferred from the cartridge 23 to the distal portion 42 of the delivery catheter 89. With the operating element 87 extending through the bore 40 in the delivery catheter 82, and the hook 86 extending distally from the delivery catheter 89 from the distal end 39 thereof, the hook 86 is engaged in the eye ring 84 of the second partitioning element 71 while the second partitioning element 71 is still in the unexpanded first state in the cartridge 23. The second partitioning element 71 is transferred from the cartridge 23 into the distal portion 42 of the delivery catheter 89 by urging the operating element 87 proximally in the bore 40 of the delivery catheter 89. With the second partitioning element 71 located in the distal bore 42 of the delivery catheter 89 and with the hook 86 engaged in the eye ring 84, the delivery catheter 89 is urged through the duodenum 2 either directly or through an endoscope extending into the duodenum 2 to the first location 4 in the duodenum 2. With the distal end 39 of the delivery catheter 89 located in the first portion 4 of the duodenum 2, the distal end 39 of the delivery catheter 89 is manoeuvred until the distal portion 42 of the bore 40 of the delivery catheter 89 is located adjacent the second location 8 in the duodenum 2 between the first and third portions 4 and 7, respectively. With the second partitioning element 71 retained in position by the hook 86 of the operating element 87, the delivery catheter 89 is urged proximally to expose and discharge the second partitioning element 71 from the delivery catheter 89. On discharge of the second partitioning element 71 from the delivery catheter 89, the carrier element 72 of the second partitioning element 71 expands from the unexpanded first state until the distal portion 79 of the carrier element 72 and the outer peripheral edge portion 82 of the isolating membrane 78 engages the inner surface 11 of the duodenum 2 to thereby substantially sealably isolate the first portion 4 of the duodenum 2 from the third portion 7 thereof. Placing and expanding the first partitioning element 70 in the first location 6 between the first portion 4 and the second portion 5 of the duodenum 2 is similar to the placing and expanding of the second partitioning element 71 in the second location 8. An endoscope is then urged through the bore 76 extending through the first partitioning element 70 and through the communicating opening 88 extending through the isolating membrane 78 into the first portion 4 of the duodenum 2, and the first portion 4 of the duodenum 2 is then insufflated by insufflating gas delivered through an insufflating gas accommodating channel of the endoscope, and the procedure is carried out by one or more instruments passed into the insufflated first portion 4 of the duodenum 2 through an instrument channel of the endoscope.

Removal of the first and second partitioning elements 70 and 71 from the duodenum 2 is substantially similar to that described with reference to the removal of the first and second partitioning elements 9 and 10 from the duodenum 2, with the exception that instead of the eye ring 84 being gripped by a gripper element, the eye ring 84 is engaged by the hook 86 of the operating element 87, and the first and second partitioning elements 70 and 71 are drawn into the distal portion 42 of the delivery catheter 89 by the operating element 87.

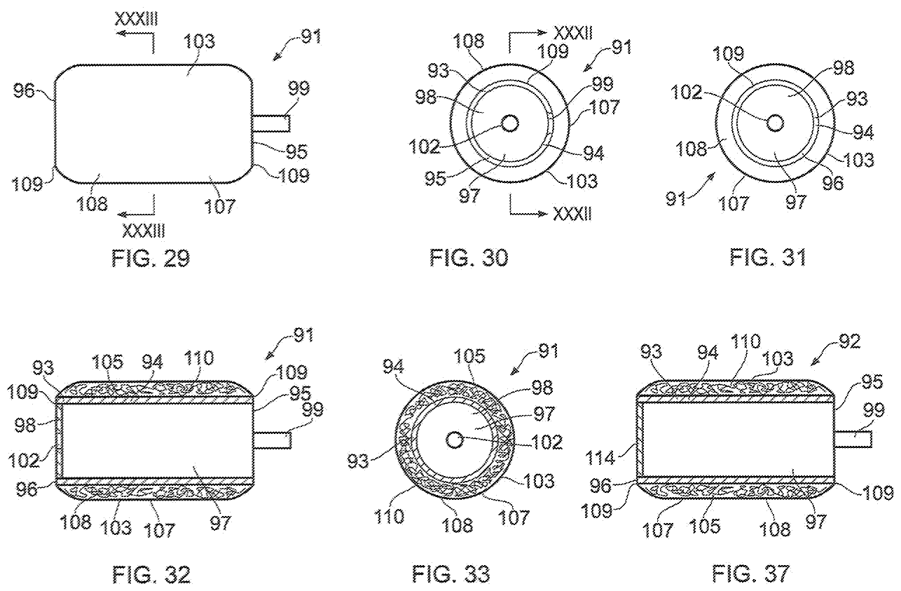

Referring now to FIGS. 29 to 37 there is illustrated a pair of partitioning elements, both of which are according to another embodiment of the invention, namely, a proximal first partitioning element 91 and a distal second partitioning element 92 for locating in a lumen, for example, the duodenum 2 described with reference to FIGS. 1 to 12 for substantially sealably isolating a portion, for example, the first portion 4 of the duodenum 2 from respective adjacent proximal second and distal third portions similar to the second and third portions 5 and 7 of the duodenum 2. In this embodiment of the invention the first and second partitioning elements 91 and 92 are substantially similar to each other, with just one exception, which will be described below, and for convenience the first partitioning element 91 will first be described.

Referring now to FIGS. 29 to 36, in this embodiment of the invention the first partitioning element 91 comprises a non-expandable carrier element 93 comprising a flexible bendable tubular member 94 of stainless-steel material extending between a proximal end 95 and a distal end 96, and having a bore 97 extending therethrough. In this embodiment of the invention the bore 97 of the tubular member 94 is closed by a membrane 98 sealably secured around the interior of the tubular member 94 and extending transversely across the tubular member 94. Typically, the membrane 98 may comprise a flexible resilient material, or may comprise a flexible non-resilient material, but impermeable or substantially impermeable to insufflating gas. A communicating opening 102 extends through the membrane 98 for sealably accommodating an instrument or an endoscope therethrough into the first portion 4 of the duodenum 2. The membrane 98 may be located anywhere along the bore 97 from the proximal end 95 to the distal end 96 of the tubular member 94, and in this embodiment of the invention is located adjacent the distal end 96 of the bore 97. However, in some embodiments of the invention the membrane 98 may be omitted, and the instrument or endoscope would be passed non-sealably through the bore 97 extending through the tubular member 94.

A connector, in this embodiment of the invention a connecting tab 99 extends from the tubular member 94 adjacent the proximal end 95 thereof. The connecting tab 99 is configured to be gripped by a gripper element 100 of an elongated operating element 101, see FIG. 37, substantially similar to the operating element 35 described with reference to FIGS. 1 to 12 for delivering and placing the first partitioning element 91 in the duodenum 2, and for removing the first partitioning element 91 therefrom.

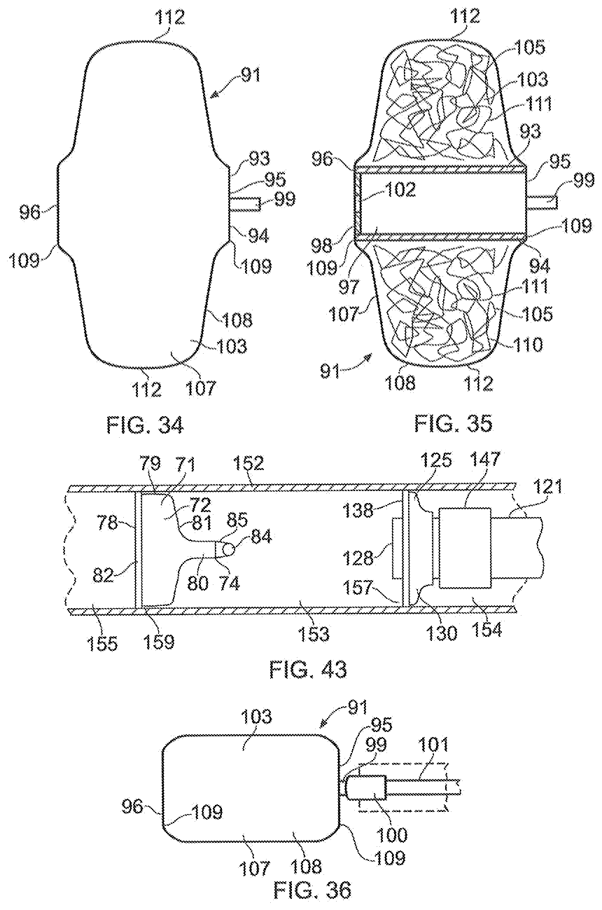

An isolating element 103 in this embodiment of the invention comprises a radially expandable isolating housing 107 extending around and secured to the tubular member 94. The isolating housing 107 comprises an expandable membrane 108 extending circumferentially around the tubular member 94 and sealably secured adjacent its opposite end edges 109 to the proximal and distal ends 95 and 96 of the tubular member 94. The expandable membrane 108 defines with the carrier element 94 an annulus 110, within which an expandable particulate material 105 is contained. In this embodiment of the invention the expandable particulate material comprises an expandable particulate material sold under the trade name Expancel by Nouryon Inc. The expandable particulate material 105 is configured to expand when heated from an unexpanded first state illustrated in FIGS. 29 to 33 with the isolating element 103 in a non-isolating state to an expanded second state illustrated in FIGS. 34 and 35 to form a rigid closed cell foam form 111 of a size up to sixty times its original unexpanded size, with the isolating element 103 in an isolating state. The expandable particulate material 105, when it expands to form the foam form 111 thereof with the isolating element 103 in the isolating state, tightly engages and may also slightly expand the duodenum 2 radially to form a substantially leak free seal with the inner surface 11 of the wall of the duodenum 2. The heating of Expancel material will be well known to those skilled in the art, and further descriptions should not be required.

The membrane 108 of the isolating element 103 in this embodiment of the invention may be rupturable in response to the expandable particulate material 105 expanding. However, in some embodiments of the invention it is envisaged that the membrane 108 may be of an elastic material, and would expand with the expandable material 105 as it expands. The expandable particulate material 105 is configured so that in the expanded second state, the expanded material 105 in the rigid foam form 111 defines an outer circumferential periphery 112 of the isolating element 103 for sealably engaging the inner surface 11 of a wall of the duodenum 2 in which the first partitioning element 91 is located.

Referring now to FIG. 37, the second partitioning element 92 as mentioned above is substantially similar to the first partitioning element 91, and similar components are identified by the same reference numerals. The only difference between the second partitioning element 92 and the first partitioning element 91 is that, in this embodiment of the invention, the membrane 98 is omitted and the bore 97 extending through the tubular member 94 is closed by a disc 114 extending transversely across the bore 97 and sealably secured to the interior of the bore 97. The disc 114 may be of any suitable material, and in this embodiment of the invention comprises a disc of stainless-steel material. However, in other embodiments of the invention, the disc 114 may be provided by a membrane similar to the membrane 98 of the first partitioning element 91, but without the communicating opening 102 extending therethrough. Like the membrane 98 of the first partitioning element 91, the disc 114 may be located in any position along the bore 97 of the tubular member 94 of the second partitioning element 92 between the proximal end 95 and the distal end 96 thereof. In this embodiment of the invention the disc 114 is located adjacent the distal end 96 of the bore 97. Otherwise, the second partitioning element 92 is similar to the first partitioning element 91.

In use, initially the second partitioning element 92 with the isolating element 103 in the unexpanded first state is delivered to the duodenum 2 adjacent the second location 8 between the first portion 4 of the duodenum 2 and the third portion 7 thereof by the operating element 101 with the gripper element 100 thereof tightly gripping the connecting tab 95. The operating element 101 may or may not be located in the bore of a delivery catheter, for example, similar to the delivery catheter 25 with the gripper element 100 extending distally from the distal end of the delivery catheter, during both placement of the second partitioning element 92 in the duodenum 2 and removal therefrom. A distal portion of a delivery catheter is illustrated in broken lines in FIG. 36.

With the second partitioning element 92 accurately located in the duodenum 2, heat is applied to the expandable particulate material 105 of the isolating element 103 for expanding thereof. The heat may be applied to the expandable particulate material 105 by any suitable heating means. For example, the gripper element 100 of the operating element 101 may comprise a heating element, which would be activated on the second partitioning element 92 being located at the second location 8 in the duodenum 2. Heat from the heating element would be conducted from the gripper element 100 through the connecting tab 99 into the tubular member 94, and in turn into the expandable particulate material 105. On expansion of the expandable particulate material 105 an outer circumferential periphery 112 of the isolating element 103 sealably engages the inner surface 11 of the duodenum 2. The gripper element 100 of the operating element 101 is disengaged from the connecting tab 99 of the second partitioning element 92, and the operating element 101 is withdrawn from the duodenum.

The first partitioning element 91 is then placed and expanded in the first location 6 of the duodenum 2 between the first and second portions 4 and 5 thereof, in a similar manner to that in which the second partitioning element 92 is placed and expanded in the second location 8 of the duodenum 2.

With the first and second partitioning elements 91 and 92 located in the first and second locations 6 and 8 in the duodenum 2 substantially sealably isolating the first portion 4 of the duodenum 2 from the second and third portions 5 and 7 thereof, an endoscope is then urged into the first portion 4 of the duodenum 2 through the bore 97 extending through the tubular member 94 and in turn through the communicating opening 102 extending through the membrane 98 of the first partitioning element 91. The first portion 4 of the duodenum 2 is then insufflated by an insufflating gas delivered through an insufflating gas accommodating channel of the endoscope, and an appropriate instrument or instruments for carrying out the procedure in the insufflated first portion 4 of the duodenum 2 are entered through an instrument channel of the endoscope into the first portion 4 of the duodenum 2.

On completion of the procedure, insufflating of the first portion of the duodenum 2 is terminated. The first partitioning element 91 is initially removed from the duodenum 2. This is carried out by withdrawing the endoscope through the communicating opening 102 in the membrane 98 and through the bore 97 in the first partitioning element 91 into the proximal second portion 5 of the duodenum 2. A suitable cutting tool, for example, a scissors or a knife or a cold or a hot wire snare is urged through the instrument channel of the endoscope to dissect the expanded isolating element 103 of the first partitioning element 91 which now comprises the expanded foam form. The dissected parts of the expanded foam isolating element 103 are collected by, for example, a forceps or a basket or other suitable device and withdrawn through the endoscope. Alternatively, the dissected parts of the isolating element 103 may be left in the lumen, if it were the duodenum 2, for normal excretion therefrom. The operating element 101 is then urged through the instrument bore of the endoscope, and the connecting tab 99 of the carrier element 93 of the first partitioning element 91 is gripped by the gripper element 100, and the carrier element 93 is removed from the lumen by withdrawing the endoscope and the operating element 101 with the carrier element 93 secured thereto from the lumen.

The endoscope is again inserted into the lumen for removing the second partitioning element 92 in a similar manner to that in which the first partitioning element 91 is removed from the lumen.

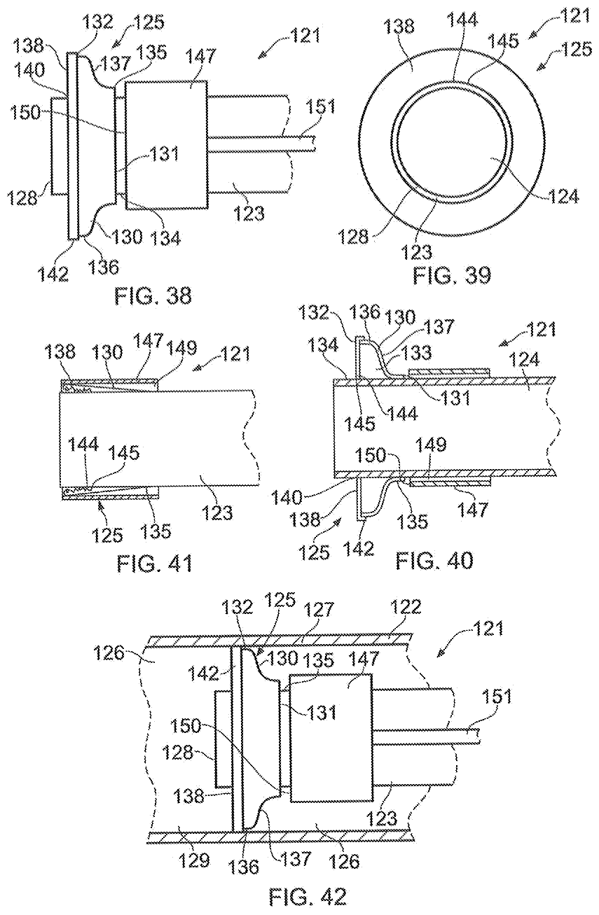

Referring now to FIGS. 38 to 42, there is illustrated an elongated delivery conduit also according to the invention and indicated generally by the reference numeral 121 for delivering instruments through a lumen, such as a lumen 122 in the body of a human or animal subject to a vessel or cavity communicating with the lumen, or to a portion of the lumen 122, in which a minimally invasive surgical or investigative procedure is to be carried out. Only a distal end portion 123 of the delivery conduit 121 is illustrated. The delivery conduit 121 has an instrument channel 124 extending therethrough for accommodating the instruments therethrough to the location, such as the vessel, cavity or the portion of the lumen in which the procedure is to be carried out, and also for accommodating insufflating gas or an insufflating gas tube for insufflating the location in which the procedure is to be carried out. The delivery conduit 121 and the instrument channel 124 are of circular transverse cross section, and the instrument channel 124 may be of a diameter at least sufficient to accommodate an endoscope therethrough. The delivery conduit 121 may be of any suitable material, for example, a polymer material, and may be rigid, semi-rigid or bendable, but typically, would be bendable in order to manoeuvre around bends in the lumen and into branches from the lumen, vessel or cavity. Ideally, the material of the delivery conduit 121 would be such that the delivery conduit 121 would maintain its transverse cross-sectional shape when bending. It is also envisaged that the material of the delivery conduit 121 may be a metal material, which ideally, would be configured to bend.

A sealing device also according to the invention and indicated generally by the reference numeral 125 is secured to the delivery conduit 121 adjacent the distal end portion 123 thereof, and is operable from a non-isolating state illustrated in FIG. 41 to an isolating state illustrated in FIGS. 38 to 40 for sealably engaging an inner surface 126 of a wall 127 of the lumen 122, for in turn sealably isolating a distal portion 129 of the lumen 122 distally of the sealing device 125 from a proximal portion 136 of the lumen 122 proximally of the sealing device 125, in order to minimise leakage of insufflating gas past the sealing device 125 from the distal portion 129 to the proximal portion 136. The sealing device 125, in general, is located on the distal end portion 123 of the delivery conduit 121 adjacent a distal end 128 thereof but spaced apart proximally from the distal end 128. In cases where the sealing device 125 is to be located towards the distal end 128 of the delivery conduit 121, typically, the sealing device 125 is located 5 mm to 30 mm proximally from the distal end 128 of the delivery conduit 121. However, it will be appreciated that the sealing device 125 may be located at any suitable location along the delivery conduit 121, and the location of the sealing device 125 on the delivery conduit 121 may be dictated by the use to which the delivery conduit 121 is to be put, and would be located on the delivery conduit 121, where the optimum sealing effect would be achieved with the inner surface 126 of the wall 127 of the lumen 122. The sealing device 125 may be releasably or non-releasably secured to the delivery conduit 121. In this embodiment of the invention the sealing device 125 is non-releasably secured to the delivery conduit 121.

The sealing device 125 comprises a carrier element 130 extending from a first end, namely, a proximal end 131 to a second end, namely, a distal end 132, and defining a bore 133 extending therethrough from the proximal end 131 to the distal end 132. The carrier element 130 is inherently configured to self-expand from an unexpanded first state illustrated in FIG. 41 to an expanded second state illustrated in FIGS. 38 to 40 when unrestrained. The carrier element 130 is of stent like construction and of lattice construction formed from a memory metal, in this embodiment of the invention nitinol. The stent construction of the carrier element 130 may be provided from a solid tube of the memory metal, which is appropriately slotted and expanded to form the lattice type construction, or may be woven from wire of memory metal. The proximal end 131 of the carrier element 130 is non-expandable and is non-releasably secured to the delivery conduit 121 around the outer periphery 134 thereof at 135. The carrier element 131 is progressively and increasingly expandable from the proximal end 131 to the distal end 132, where the carrier element 130 defines an outer peripheral portion 136. An intermediate portion 137 of the carrier element 130 extending from the proximal end 131 to the distal end 132 is progressively expandable. An isolating element, in this embodiment of the invention an isolating membrane 138 is mounted on the carrier element 130 and is non-releasably and sealably secured to the delivery conduit 121 around the outer periphery 134 thereof at 140. The isolating membrane 138 is operable from a non-isolating state illustrated in FIG. 41, which corresponds to the non-isolating state of the sealing device 125, to an isolating state illustrated in FIGS. 38 to 40, which corresponds to the isolating state of the sealing device 125, as the carrier element 130 expands from the first state to the second state. The isolating membrane 138 defines an outer peripheral portion 142, which is secured to the outer peripheral portion 136 of the carrier element 130, so that when the carrier element 130 expands from the first state to the second state, the outer peripheral portion 142 of the isolating membrane 138 is urged into sealable engagement with the inner surface 126 of the wall 127 of the lumen 122 to minimise leakage of insufflating gas from the distal portion 129 of the lumen 122 when insufflated, to the proximal portion 136 thereof.

A circular opening 144 extends centrally through the isolating membrane 138 for accommodating the delivery conduit 121 therethrough. The circular opening 144 defines an inner rim 145 around which the isolating membrane 138 is sealably secured to the outer periphery 134 of the delivery conduit 121 at 140 by a suitable securing means, for example, by an adhesive, or by heat welding or other suitable means.

The isolating membrane 138 may be of any suitable flexible material, and may be of an expandable material or a non-expandable material. In this embodiment of the invention the isolating membrane 138 comprises a cloth material coated with a suitable coating which is substantially impermeable to insufflating gas, so that when coated with the coating material, the isolating membrane 138 is substantially impermeable to insufflating gas. By being of a flexible material, the isolating membrane 138 is foldable into a folded state within the carrier element 130 when the carrier element 130 is in the unexpanded first state, as illustrated in FIG. 41.

A retaining means, which may comprise a retaining ring or a retaining sleeve, is provided extending around the sealing device 125 for retaining the carrier element 130 in the unexpanded first state, and in turn, for retaining the sealing device 125 in the non-isolating state. In this embodiment of the invention the retaining means comprises an elongated retaining sleeve 147 having a bore 149 extending therethrough, through which the delivery conduit 121 extends. The diameter of the bore 149 of the retaining sleeve 147 is such as to engage and retain the carrier element 130 in the unexpanded first state with the isolating membrane 138 folded within the carrier element 130 and with the carrier element 130 located between the retaining sleeve 147 and the delivery conduit 121.

The retaining sleeve 147 is manually slideable longitudinally on the delivery conduit 121, and is slideable proximally from a retaining state illustrated in FIG. 41 with the sealing device 125 within the bore 149 of the retaining sleeve 147 for retaining the carrier element 130 in the unexpanded first state therein and the isolating membrane 138 in the folded non-isolating state, to a release state illustrated in FIGS. 38 to 40 with a distal end 150 of the retaining sleeve 147 located spaced apart proximally of the sealing device 125 to permit expansion of the carrier element 130 from the unexpanded first state to the expanded second state for urging the isolating membrane 138 to the isolating state.

An actuating means, in this case, an elongated actuating member 151 extends proximally from the retaining sleeve 147 along the delivery conduit 121 to the proximal end (not shown) thereof for operating the retaining sleeve 147 between the retaining state and the release state. Alternatively, the retaining sleeve 147 may extend proximally along the delivery conduit 121 to the proximal end thereof.

On the other hand, if the retaining means were provided by a carrier ring, the carrier ring would be of sufficient width to engage the carrier element 130 adjacent the distal end 132 thereof in order to retain the carrier element 130 in the unexpanded first state with the isolating membrane 138 folded within the carrier element 130.

In use, with the retaining sleeve 147 in the retaining state as illustrated in FIG. 41 retaining the carrier element 130 in the unexpanded first state and the isolating membrane 138 folded within the carrier element 130, the delivery conduit 121 is inserted through a suitable orifice, for example, orally, transanally, transvaginally or through any other suitable bodily orifice or incision into the relevant lumen 122. The delivery conduit 121 with the retaining sleeve 147 in the retaining state is urged through the lumen 122 to the location in which the procedure is to be carried out and which is to be insufflated, The location in which the procedure is to be carried out as discussed above may be a portion of the lumen 122 such as the distal portion 129 thereof, or a vessel or cavity to which the lumen 122 extends. When the delivery conduit 121, with the retaining sleeve 147 in the retaining state, has been urged through the lumen 122 with the distal end 128 of the delivery conduit 121 in the lumen 122 adjacent the proximal end of the location at which the procedure is to be carried out, the retaining sleeve 147 is urged proximally on the delivery conduit 121 from the retaining state to the release state by operating the actuating member 151, thereby releasing the carrier element 130 to self-expand from the unexpanded first state to the expanded second state until the outer peripheral portion 136 of the carrier element 130 and the outer peripheral portion 142 of the isolating membrane 138 sealably engage the inner surface 126 of the wall 127 of the lumen 122, see FIG. 42. With the isolating membrane 138 sealably engaging the lumen 122, the sealing device 125 sealably isolates the distal portion 129 of the lumen 122 from the proximal portion 136 thereof, so that the distal portion 129 may be insufflated without insufflating gas leaking from the distal portion 129 to the proximal portion 136. An endoscope (not shown) is urged through the instrument bore 124 of the delivery conduit 121 into the distal portion 129 of the lumen, or through the distal portion 129 into a vessel or cavity in which the procedure is to be carried out. The distal portion 129 of the lumen 122, or the vessel or cavity, as the case may be, is then insufflated by insufflating gas delivered either through the endoscope, or through a separate gas delivery tube extending through the instrument bore 124 of the delivery conduit 121, or directly through the instrument bore 124.

One or more instruments for carrying out the procedure are then urged through an instrument channel of the endoscope to the insufflated location, or directly through the instrument bore 124 of the delivery conduit 121.

On completion of the procedure in the distal portion 129 of the lumen 122 or in the vessel or cavity, the endoscope and other instruments and tubes are withdrawn through the instrument bore 124 of the delivery conduit 121 and insufflating of the distal portion 129 of the lumen 122, or the vessel or cavity is terminated. The retaining sleeve 147 is urged distally on the delivery conduit 121 from the release state to the retaining state by the actuating member 151 for urging the carrier element 130 from the expanded second state to the unexpanded first state. As the retaining sleeve 147 is being urged distally from the release state to the retaining state, the distal end 150 of the retaining sleeve 147 engages the carrier element 130 adjacent the intermediate portion 137 thereof, and further urging of the retaining sleeve 147 distally into the retaining state results in the carrier element 130 being progressively urged from the expanded second state to the unexpanded first state with the isolating membrane 138 being folded within the carrier element 130. Once the retaining sleeve 147 has been urged into the retaining state, the delivery conduit 121 is withdrawn from the lumen 122.

Referring now to FIG. 43, when a procedure is to be carried out in a portion of a lumen 152, such as a first portion 153 thereof, and the first portion 153 is to be insufflated, and sealably isolated from respective proximal and distal second and third portions 154 and 155 thereof to the proximal and distal ends 157 and 159, respectively, of the first portion 153, the delivery conduit 121 may be used in conjunction with any one of the second partitioning elements 10, 62, 71 or 92 described with reference to FIGS. 1 to 37. In which case, with the retaining sleeve 147 in the retaining state, the delivery conduit 121 is urged into the lumen 152 from a proximal end thereof until the distal end 128 is located in the lumen 122 adjacent the proximal end 157 of the first portion 153 of the lumen 152. The retaining sleeve 147 is then urged from the retaining state to the release state, thereby releasing the carrier element 130 from the unexpanded first state to expand to engage the lumen 152 adjacent the proximal end 157 of the first portion 153 thereof, thereby urging the isolating membrane 138, and in turn the sealing device 125 into the isolating state to substantially sealably isolate the first portion 153 of the lumen 152 from the proximal portion 154 thereof.

The selected one of the second partitioning elements 10, 62, 71 or 92 is then delivered through the instrument bore 124 of the delivery conduit 121 by the delivery catheter 25 or the operating element 101, as the case may be, and is placed and expanded at the distal end 159 of the first portion 153 of the lumen 122.

The first portion 153 is then insufflated through the instrument bore 124 of the delivery conduit 121 or through an endoscope extending through the instrument bore 124 of the delivery conduit 121, and the minimally invasive procedure is then carried out in the first portion 153 of the lumen 152.

While in general, the partitioning elements and the sealing device have been described for substantially sealably isolating a portion of a lumen to be insufflated from another portion of the lumen not to be insufflated, the degree of sealing achieved by each partitioning element and the sealing device will depend on the lumen in which the partitioning element and the sealing device is being deployed. In some lumens, while the outer peripheral portion of the isolating membrane will engage the inner surface of the lumen adjacent the location of the lumen at which sealing is to be achieved, due to the nature of some lumens in the human or animal body, some leakage, although in general minimal may occur across the boundary between the partitioning element or the sealing device and the inner surface of the lumen. Accordingly, while in general, it is desirable that a total seal be achieved between each partitioning element and/or the sealing device and the inner surface of the wall of the lumen in which the partitioning element and/or the sealing device is deployed, due to the nature of lumens in the body of a human or animal subject, a total seal may not always be achievable. However, in general the seal between each partitioning element and/or the sealing device and the inner surface of the lumen will be such that the leakage of insufflating gas across the boundary between the partitioning element or the sealing device and the inner surface of the lumen will be minimal.

Additionally, it will be appreciated that while it is desirable that the isolating membrane adjacent the communicating opening therethrough should sealably engage an instrument or endoscope extending therethrough, it may not always be possible to achieve a total seal between the isolating membrane and the instrument or endoscope extending through the communicating opening. However, in general, the leakage of insufflating gas through the communicating opening will be minimised.

While the isolating membrane and the lanyards comprising the connecting members of the partitioning elements described with reference to FIGS. 1 to 18 have both been described as being located at the proximal end of the carrier element, in some embodiments of the invention it is envisaged that with the connecting members located adjacent the proximal end of the carrier element, the isolating membrane may be located at the distal end of the carrier element, or may be located extending transversely in the bore extending through the carrier element anywhere between the proximal and distal ends thereof.

While in the embodiments of the invention described the connector has been described as comprising a connecting tab which is separate from the operating element, in some embodiments of the invention it is envisaged that the connector may form a part of the operating element, for example, the distal end of the operating element may be connected directly to the isolating membrane or to the lanyards comprising the connecting members, and such an arrangement could be a practical arrangement, particularly, from the point of view of the proximal first partitioning element.

While the carrier elements of the first and second partitioning elements and of the sealing device have been described as being of a woven construction of memory metal, it is envisaged that in some embodiments of the invention the carrier elements may be constructed from a slotted tubular member of a memory metal, which would be expanded to the second state of the second transverse cross-sectional area and temperature set in the second state. The expanded carrier element would then be compressed to the first state of the first transverse cross-sectional area.

While the isolating membranes have been described as being of a specific material, any other suitable material may be used. However, the material of the isolating membrane should be of a material which is impermeable or substantially impermeable to the insufflating gas, or which at least has a relatively low permeability to the insufflating gas.

While the carrier elements have been described as being of stent type construction, the carrier elements may be of any other suitable construction or material. For example, the carrier elements may be of a spring type material, which would be maintained in an unexpanded first state by a retaining means, such as the cartridge or other such retaining sleeve, and when released from the retaining means would spring outwardly to an expanded second state thereof of greater diameter than the diameter of the carrier element in the first state. It is also envisaged that the carrier elements may be of memory metal, which would be stable in the first state thereof at normal room temperature, but on being exposed to body temperature would expand from the first state to the second state. It is also envisaged that the carrier elements may comprise of material which would be expandable, for example, by inflating a balloon of a balloon catheter located within the bore of the carrier element, to engage the inner surface of the wall of the lumen, vessel or cavity to be insufflated, and on being expanded by inflating the balloon to engage the wall of the lumen to be insufflated, the carrier element would be stretched beyond its elastic limit, so that it would be stable in the expanded state.

While the first and second partitioning elements and the sealing device have been described for sealably isolating a first portion of the passageway of the duodenum from a second and/or a third adjacent portion thereof, it will be readily apparent to those skilled in the art that the first and second partitioning elements and the sealing device may be used for sealably isolating a first portion of any lumen, vessel, cavity or organ from a second portion and/or a third portion thereof.

It is also envisaged in some embodiments of the invention a single partitioning element only may be provided for partitioning a first portion of a lumen from a second portion, and in general, it is envisaged that the first portion would be located proximally relative to the second portion.

While in the embodiments of the invention described the first partitioning elements have been described as being withdrawn from the lumen prior to withdrawal of the corresponding ones of the second partitioning elements, in some embodiments of the invention, it is envisaged that the second partitioning element may be withdrawn from the lumen prior to withdrawal of the first partitioning element. In which case, it is envisaged that the endoscope would be urged into the first portion of the lumen through the communicating opening in the isolating membrane of the first partitioning element for withdrawing the second partitioning element from the lumen through the first partitioning element prior to withdrawal of the first partitioning element. Similarly, it is also envisaged that the first partitioning elements may be placed in the lumen prior to the placing of the second partitioning elements.

While the delivery catheter has been described as being urged through an instrument bore of an endoscope, in some embodiments of the invention it is envisaged that a delivery catheter may be urged into the duodenum or other lumen in which one or pair of the partitioning elements are to be deployed through an elongated tubular member which may be releasably secured to an external surface of the endoscope and which would extend longitudinally along the endoscope to the distal end thereof. Such a tubular member releasably secured to an endoscope by suitable clips is disclosed in PCT Published Application No. WO 2021/209983 of the present applicant. Additionally, or alternatively, such a tubular member may be used for insufflating the portion of the lumen to be insufflated, and/or may also be used for accommodating an instrument into the portion of the lumen being insufflated and in which the procedure is being carried out.

While the sealing device has been described as being located on a delivery conduit in the embodiment of the invention described with reference to FIGS. 38 to 41, it is envisaged that the sealing device may be located on any instrument, and typically, would be located adjacent the distal end of such an instrument. For example, it is envisaged that the sealing device may be located releasably or non-releasably on an endoscope, typically, towards the distal end of the endoscope for sealably locating the endoscope in a lumen, vessel or cavity, or the sealing device may be located on a surgical or investigative instrument for sealably locating the instrument in a lumen, vessel or cavity.

While the partitioning elements and the delivery conduit and the delivery catheter as well as the operating element and the components thereof have been described as being of specific materials, it is envisaged that any other suitable materials may be used. In general, although not necessarily, it is envisaged that the materials thereof would be biocompatible materials.

While the partitioning elements have been described as being delivered to a site in the lumen, vessel or cavity in which they are to be located by or through a delivery catheter or an endoscope, the partitioning elements may be delivered to the site by or through any other suitable delivery means.

In some embodiments of the invention it is envisaged that the partitioning elements and the sealing device may be configured when expanded from the unexpanded first state to the expanded second state to engage the wall of the lumen vessel or cavity such that the wall of the lumen vessel or cavity is radially expanded, in order to enhance sealing between the partitioning element or the sealing device and the wall of the lumen, vessel or cavity.

While the sealing device and the delivery conduit and the partitioning elements have been described for sealably isolating a first portion of a lumen from a second portion and/or a third portion thereof whereby the portion to be sealably isolated is to be sealably isolated for the purpose of insufflating the first portion thereof, it is envisaged that the sealing device and the delivery conduit and the partitioning elements may be used for sealably isolating a portion in a lumen from another portion thereof, or for sealably isolating a cavity or a vessel for purposes other than for insufflating the relevant portion of the lumen, the vessel or cavity.

It is also envisaged that the delivery conduit may be provided with a sealing means located in the instrument bore thereof for sealably engaging an endoscope or other instrument extending therethrough. Such a sealing means may, for example, comprise a membrane or a valve, such as a duckbill valve.

While the partitioning elements of the embodiment described with reference to FIGS. 13 to 18 have been described as comprising four lanyards provided by respective connecting members connecting the connecting tab to the proximal end of the corresponding carrier element, any number of lanyards may be provided. In some embodiments of the invention it is envisaged that a pair of lanyards may be sufficient. Needless to say, the lanyards may be provided by any suitable flexible connecting members.

While two partitioning elements have been described for sealably isolating a portion of the lumen to be isolated from the remainder of the lumen for insufflating thereof, it is envisaged that in some embodiments of the invention only a single partitioning element will be required. For example, if the portion of the lumen to be sealably isolated from the remainder of the lumen is located towards an end of the lumen, and that end of the lumen is naturally sealed by, for example, a sphincter, the portion of the lumen to be sealably isolated could be sealably isolated from the remainder of the lumen by a single one of the partitioning elements located in the lumen spaced apart from the sphincter to define with the sphincter the portion of the lumen to be insufflated and sealably isolated from the remainder of the lumen. In which case, if the portion of the lumen to be insufflated between the sphincter and the single partitioning element could be accessed by an endoscope through the sphincter, then the partitioning element would be a partitioning element similar to any of the second partitioning elements. Otherwise, if it were necessary to access the portion to be insufflated between the sphincter and the partitioning element, through the partitioning element, then the partitioning element would have to be provided by one of the first partitioning elements, so that the endoscope or other instruments could be entered into the portion to be insufflated between the sphincter and the partitioning element through the communicating opening of the first partitioning element.

It will be appreciated that other suitable connectors besides a connecting tab or an eye ring may be provided for attaching the carrier element to the operating element.

It will also be appreciated that the carrier elements of the partitioning elements of the described with reference to FIGS. 29 to 37 may be of any other suitable material besides stainless-steel material. It will also be appreciated that in some embodiments of the invention the isolating element of the partitioning elements described with reference to FIGS. 29 to 37 may be provided by other suitable expandable materials besides Expancel material, and such materials may be sensitive to other means for expanding thereof besides being heat sensitive.

While the partitioning elements, sealing device and methods have been described for sealably isolating a portion of the duodenum from the remainder of the duodenum, it will be readily apparent to those skilled in the art that the partitioning elements, sealing device and the method for sealably isolating a portion of the duodenum may be used for sealably isolating any portion in any vessel or cavity or any portion of any other lumen, for example, the colon, the large intestine, the oesophagus, the bronchial tube, an artery, vein or any other lumen in the human or animal body.

While the partitioning elements and the sealing device, as well as the carrier elements and the isolating elements thereof have been described as being of circular transverse cross-section, the partitioning elements, the sealing device, the carrier elements and the isolating elements may be of any suitable transverse cross-section, and in general, the partitioning elements, the sealing device as well as the carrier elements and the isolating elements will be of a sufficiently flexible and compliant material to substantially take-up the transverse cross-sectional shape of the lumen when expanded from the unexpanded first state thereof.

1. A partitioning element for isolating a first portion of an elongated lumen from a second portion thereof, the partitioning element comprising:
an expandable carrier element for locating in the lumen between the first and second portions thereof, the carrier element extending from a proximal end to a distal end and having a bore extending longitudinally therethrough and being radially expandable from a first state defining a first transverse cross-sectional area to a second state defining a second transverse cross-sectional area greater than the first transverse cross-sectional area,
an isolating element carried on and secured to the carrier element and being urgeable from a non-isolating state to an isolating state with one of the isolating element and the carrier element engaging an inner surface of the lumen and the isolating element extending transversely across the lumen to substantially sealably isolate the first portion of the lumen from the second portion thereof as the carrier element expands from the first state thereof to the second state, and
a connector adapted to be releasably or non-releasably connectable with an operating element for withdrawing the partitioning element from the lumen, the connector being formed by a part of the carrier element or by a part of the isolating element or being connected either to the isolating element or to at least two lanyards secured to the periphery of the carrier element at circumferentially spaced apart intervals adjacent the proximal end thereof.

2. A partitioning element as claimed in claim 1 in which the isolating element is secured to the carrier element adjacent one of the proximal end of the carrier element, the distal end of the carrier element and a location intermediate the proximal and distal ends of the carrier element.

3. A partitioning element as claimed in claim 1 in which the isolating element is secured to the carrier element adjacent a periphery of the carrier element and is engageable with an inner surface of the lumen with substantially sealable engagement.

4. A partitioning element as claimed in claim 1 in which one or both of the isolating element and the at least two lanyards are secured to the carrier element, so that when the distal end of the carrier element is engaged in the lumen, by urging the connector proximally the transverse cross-sectional area defined by the carrier element commences to reduce from the proximal end thereof towards the distal end thereof.

5. A partitioning element as claimed in claim 1 in which the connector comprises a part of the isolating element adjacent a central portion thereof.

6. A partitioning element as claimed in claim 1 in which the connector comprises one of a connecting loop, a connecting ring and a connecting tab.

7. A partitioning element as claimed in claim 1 in which a communicating opening is formed in the isolating element to accommodate an instrument or an insufflating gas delivery conduit therethrough to the first portion of the lumen.

8. A pair of the partitioning elements as claimed in claim 1 in which a first one of the partitioning elements is adapted for locating between the first and the second portions of the lumen and a second one of the partitioning elements is adapted for locating in the lumen spaced apart from the first one of the partitioning elements at a location between the first portion and a third portion of the lumen for substantially sealably isolating the first portion of the lumen from the third portion thereof.

9. A method for isolating a first portion of an elongated lumen from a second portion thereof, the method comprising:
providing a partitioning element comprising:
an expandable carrier element extending from a proximal end to a distal end and having a bore extending longitudinally therethrough and being radially expandable from a first state defining a first transverse cross-sectional area to a second state defining a second transverse cross-sectional area greater than the first transverse cross-sectional area,
an isolating element carried on and secured to the carrier element and being urgeable from a non-isolating state to an isolating state as the carrier element expands from the first state thereof to the second state thereof, and
a connector formed by a part of the carrier element or by a part of the isolating element or being connected either to the isolating element or to at least two lanyards secured to the periphery of the carrier element at circumferentially spaced apart intervals adjacent the proximal end thereof, the method further comprising:
placing the partitioning element in the lumen at a location between the first and second portions thereof with the carrier element in the first state thereof and the proximal end of the carrier element located adjacent the second portion of the lumen, and
radially expanding the carrier element from the first state thereof until the one of the isolating element and the carrier element engages an inner surface of the lumen with the isolating element extending transversely across the lumen substantially sealably isolating the first portion thereof from the second portion thereof.

10. A method as claimed in claim 9 further comprising delivering the partitioning element by a delivery element to the location in the lumen between the first and second portions thereof with the partitioning element located in the delivery element, and discharging the partitioning element from the delivery element.

11. A method as claimed in claim 10 further comprising retaining the partitioning element at the location in the lumen between the first portion and the second portion thereof by an operating element connected to the connector of the partitioning element, as the partitioning element is being discharged from the delivery element.

12. A method as claimed in claim 10 in which the partitioning element is discharged from the delivery element by urging the delivery element proximally in the lumen relative to the partitioning element.

13. A method as claimed in claim 11 in which the partitioning element is drawn proximally into the delivery element by the operating element with the operating element connected to the connector for removal of the partitioning element from the lumen.

14. A method as claimed in claim 13 in which the operating element is urged progressively proximally into the delivery element for reducing the transverse cross-sectional area of the carrier element from the proximal end thereof towards the distal end thereof.

15. A method as claimed in claim 9 in which a pair of the partitioning elements are placed in the lumen to isolate the first portion of the lumen from the second portion thereof and from a third portion thereof remote from the second portion thereof, a first one of the partitioning elements being located in the lumen between the first and second portions thereof for isolating the first portion of the lumen from the second portion thereof, and a second one of the partitioning elements being located between the first portion thereof and the third portion thereof for isolating the first portion of the lumen from the third portion thereof.

16. A method for insufflating a first portion of a lumen in which a minimally invasive procedure is to be carried out without insufflating an adjacent second portion of the lumen, the method comprising:
providing a partitioning element expandable radially from a first state defining a first transverse cross-sectional area to a second state defining a second transverse cross-sectional area greater than the first transverse cross-sectional area,
placing the partitioning element in the first state thereof in the lumen between the first portion thereof and the second portion thereof,
radially expanding the partitioning element from the first state thereof until the partitioning element engages an inner surface of the lumen with the partitioning element extending transversely across the lumen substantially sealably isolating the first portion thereof from the second portion thereof, and
insufflating the first portion of the lumen by an insufflating gas.

17. A method as claimed in claim 16 in which the partitioning element comprises:
an expandable carrier element extending from a proximal end to a distal end, and having a bore extending longitudinally therethrough and being radially expandable from a first state defining the first transverse cross-sectional area to the second state defining the second transverse cross-sectional area,
an isolating element carried on and secured to the carrier element and being urgeable from a non-isolating state to an isolating state for substantially sealably isolating the first portion of the lumen from the second portion thereof, the isolating element being urgeable from the non-isolating state to the isolating state as the carrier element expands from the first state to the second state, and
a connector formed by a part of the carrier element or by a part of the isolating element, or being connected either to the isolating element or to at least two lanyards secured to the periphery of the carrier element at circumferentially spaced apart intervals adjacent the proximal end thereof.

18. A method as claimed in claim 16 in which a communicating opening extends through the partitioning element, and insufflating gas is delivered to the first portion of the lumen through the communicating opening.

19. A method as claimed in claim 16 in which a pair of the partitioning elements are provided for isolating the first portion of the lumen from the second portion thereof and from a third portion of the lumen remote from the second portion thereof without insufflating either the second or the third portion of the lumen, a first one of the partitioning elements is placed in the lumen at a location between the first and second portions thereof, and a second one of the partitioning elements is placed in the lumen at a location between the first and third portions thereof for isolating the first portion of the lumen from the third portion thereof.

20. A method as claimed in claim 19 in which the first and second ones of the partitioning elements are sequentially placed in the lumen.

21. A lumen comprising a first portion thereof and a second portion thereof substantially sealably isolated from the first portion thereof by a partitioning element as claimed in claim 1.
